(19) United States
(12) Patent Application Publication
(10) Pub. No.: US 2026/0285837 A1
(43) Pub. Date: Sep. 24, 2026

(54) CHECKPOINT KINASE 1 (CHK1) INHIBITOR CRYSTALLINE FORM AND USES THEREOF

(71) Applicant: Boundless Bio, Inc., San Diego, CA (US)

(72) Inventors: Eric F. CONNOR, San Jose, CA (US); Stephen Todd MEYER, San Diego, CA (US); Anthony B. PINKERTON, Rancho Santa Fe, CA (US)

(21) Appl. No.: 19/471,073

(22) PCT Filed: Apr. 2, 2024
(86) PCT No.: PCT/US2024/022614

§ 371 Date: Sep. 30, 2025

Related U.S. Application Data
(60) Provisional application No. 63/493,846, filed on Apr. 3, 2023.

(51) Int. Cl. C07D 401/14; A61K 31/497; A61K 31/506; A61K 31/517; A61K 31/519; A61K 31/5377

(52) U.S. Cl. CPC C07D 401/14; A61K 31/497; A61K 31/506; A61K 31/517; A61K 31/519; A61K 31/5377

(57) ABSTRACT

Provided herein is a pharmaceutical composition (1) comprising a CHK1 inhibitor.

### CROSS-REFERENCE

This application claims the benefit of U.S. Provisional Application Ser. No. 63/493,846 filed Apr. 3, 2023 which is hereby incorporated by reference in its entirety.

### BACKGROUND OF THE INVENTION

Cancer remains the second leading cause of death in the United States (US), accounting for approximately 1,900,000 new diagnoses and 610,000 deaths on an annual basis. Globally, cancer is the second leading cause of death, and was responsible for nearly 10 million deaths in 2020; nearly 1 in 6 deaths was due to cancer. The number of new cases is expected to rise by 70% over the next 2 decades.

In solid malignancies, metastatic spread, and systemic disease accounts for approximately 90% of cancer-related deaths. Despite progress made over the past few decades, there remains a need for the development of targeted interventions for advanced or metastatic solid tumors.

Patients with cancers that harbor oncogene amplifications, with the exception of HER2 (or ERBB2), have no targeted therapies approved as standard of care. Generally, these patients suffer worse survival rates than patients with other forms of oncogene alteration or with no known oncogene alteration.

There are currently no therapies approved to treat patients with these other oncogene-amplified tumors, and especially no oral therapy comprising a stable pharmaceutical form of a CHK1 inhibitor.

### BRIEF SUMMARY OF THE INVENTION

Disclosed herein is a crystalline form of 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

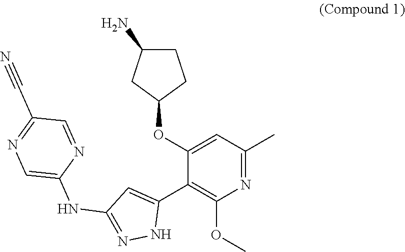 [SMILES: COc1nc(C)cc(O[C@@H]2CC[C@H](N)C2)c1-c1cc(Nc2cnc(C#N)cn2)n[nH]1]

or a pharmaceutically acceptable salt or solvate thereof.

Also disclosed herein is a crystalline form of freebase 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

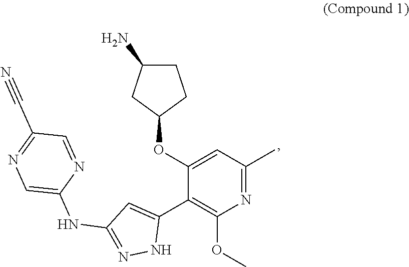 [SMILES: COc1nc(C)cc(O[C@@H]2CC[C@H](N)C2)c1-c1cc(Nc2cnc(C#N)cn2)n[nH]1]

or a pharmaceutically acceptable solvate thereof.

Also disclosed herein is a crystalline form of anhydrous freebase 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

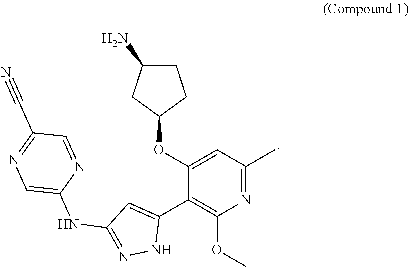 [SMILES: COc1nc(C)cc(O[C@@H]2CC[C@H](N)C2)c1-c1cc(Nc2cnc(C#N)cn2)n[nH]1]

In some embodiments, the crystalline Compound 1 is freebase Form FB-1 characterized as having at least one of the following properties:
(a) an X-ray powder diffraction (XRPD) pattern substantially the same as shown in FIG. 1;
(b) an X-ray powder diffraction (XRPD) pattern with characteristic peak at 11.96±0.1° 2θ, 18.81±0.1° 2θ, 19.91±0.1° 2θ, 21.67±0.1° 2θ, and 26.75±0.1° 2θ;
(c) an X-ray powder diffraction (XRPD) pattern with characteristic peak at 13.87±0.1° 2θ and 17.06±0.1° 2θ;
(d) a Differential Scanning Calorimetry (DSC) thermogram with an endotherm having a peak temperature at about 216.5° C. (onset);
(e) a Thermogravimetric Thermal Analysis (TGA) thermogram substantially the same as shown in FIG. 2;
(f) a Thermogravimetric Thermal Analysis (TGA) thermogram exhibiting a mass loss of about 0.10% from the onset of heating up to approximately 100.0° C.; or
(g) combinations thereof.

Also disclosed herein is a pharmaceutical composition comprising a crystalline form disclosed herein and a pharmaceutically acceptable excipient.

Also disclosed herein is a method of treating cancer in a subject in need thereof comprising administering to the subject a crystalline form disclosed herein.

In some embodiments, the cancer comprises a solid tumor.

In some embodiments, the cancer comprises a locally advanced or metastatic non-resectable solid tumor.

In some embodiments, the cancer comprises a tumor or tumor cells harboring an oncogene amplification.

In some embodiments, the oncogene amplification comprises an amplification of ABL, AKT1, AKT2, ALK, androgen receptor, BRAF, CCND1, CCND2, CCND3, CCNE1, CDK12, CDK4, CDK6, EGFR, ERBB2, EZH2, FGFR1, FGFR2, FGFR3, FLT3, IDH1/2, JAK2, JAK3, KIT, KRAS, MDM2, MDM4, MET, MYC, MYCL, MYCN, NRAS, PDGFRA, TERT, VEGFRA, or any combination thereof.

In some embodiments, the oncogene amplification resides on ecDNA.

In some embodiments, the oncogene amplification resides on one or more chromosomal loci.

In some embodiments, the oncogene amplification is an ecDNA-derived amplification.

In some embodiments, the cancer is an ovarian cancer.

In some embodiments, the ovarian cancer is a platinum resistant high-grade serous ovarian cancer, a primary peritoneal cancer, or a fallopian tube cancer.

In some embodiments, the cancer is a uterine cancer.

In some embodiments, the uterine cancer is a high-grade endometrial carcinoma, a uterine serous carcinoma or a uterine carcinosarcoma.

In some embodiments, the cancer is colorectal cancer, esophageal cancer, gastric cancer, gastroesophageal junction (GEJ) cancer, head and neck squamous cell carcinoma, liposarcoma, non-small cell lung cancer, or subtype squamous cell carcinoma.

In some embodiments, the cancer is a neuroblastoma.

In some embodiments, the cancer is breast cancer, cholangiocarcinoma, esophageal cancer, neck squamous cell carcinoma, non-small cell lung cancer, stomach cancer, or subtype squamous cell carcinoma.

In some embodiments, the cancer is esophageal cancer, non-small cell lung cancer, a sarcoma, or stomach cancer.

In some embodiments, the treatment further comprises administering an additional therapeutic agent.

In some embodiments, the oncogene amplification comprises CDK4, CDK6, EGFR, FGFR1, FGFR2, or FGFR3.

In some embodiments, the treatment further comprises administering a CDK4/6 inhibitor, an EGFR inhibitor, or a FGFR inhibitor. In some embodiments, the EGFR inhibitor is erlotinib. In some embodiments, the FGFR inhibitor is pemigatinib. In some embodiments, the FGFR inhibitor is futibatinib. In some embodiments, the CDK4/6 inhibitor is abemaciclib.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the X-Ray Powder Diffraction (XRPD) pattern of Compound 1 freebase Form FB-1.

FIG. 2 shows the Thermogravimetric Analysis (TGA) thermogram and Differential Scanning Calorimetry (DSC) thermogram of Compound 1 freebase Form FB-1.

FIG. 3 shows the X-Ray Powder Diffraction (XRPD) pattern of Compound 1 freebase Form FB-2.

FIG. 4 shows the X-Ray Powder Diffraction (XRPD) pattern of Compound 1 freebase Form FB-3.

FIG. 5 shows the X-Ray Powder Diffraction (XRPD) pattern of Compound 1 freebase Form FB-4.

FIG. 6 shows the X-Ray Powder Diffraction (XRPD) pattern of Compound 1 freebase Form FB-5.

### DETAILED DESCRIPTION OF THE INVENTION

Focal high-copy number oncogene amplifications are frequently observed to occur on extrachromosomal DNA (ecDNA). ecDNA are found in tumor cells and are derived from extrachromosomal fragments of genomic DNA that often encode full length genes and regulatory regions such as promoters. ecDNA may be found as physically distinct from chromosomes and are engendered with unique properties, including an open chromatin architecture associated with hyper-transcription and a predilection for structural variation. In addition, because ecDNA lack centromeres, extrachromosomally located ecDNA are inherited during cellular division via acentric, non-Mendelian segregation, which enables high copy number gene heterogeneity across the tumor cell population. Due to these properties, ecDNA are a common cellular mechanism for oncogene amplification (e.g., EGFR), and they facilitate hyper-transcription and overexpression of oncoproteins, which drive tumor growth and survival. Moreover, these features afford oncogene amplified ecDNA-enabled tumor cells with unparalleled genomic plasticity, facilitating both oncogenesis and circumvention of therapeutic pressure through rapid genomic evolution. Cancer cells that harbor oncogene amplifications on ecDNA bear high levels of intrinsic DNA replication stress (RS). The checkpoint kinase 1 (CHK1) serves an essential role in managing RS, making CHK1 a potential therapeutic target for cancers that have intrinsic elevated RS, including those with ecDNA-enabled oncogenic amplifications. Consistent with this hypothesis, ecDNA-enabled oncogene-amplified tumor cells display enhanced sensitivity to CHK1 inhibition when compared to ecDNA negative non-amplified cells. Applying targeted therapy (e.g., EGFR inhibitor) pressure to the protein products of oncogenes (e.g., EGFR) amplified on ecDNA induces cancer cells to evade such pressure, and these resistance mechanisms further increase RS and reliance upon CHK1. Accordingly, combining targeted therapy pressure (e.g., EGFR inhibitor) with CHK1 pressure (i.e., CHK1 inhibitor) in ecDNA-enabled tumor cells provides a synergistic therapeutic effect. There is a need therefore, as recognized and addressed herein, to provide CHK1 inhibitors with desired clinical and therapeutic properties, including stable pharmaceutical forms of such CHK1 inhibitors.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms “treat,” “treated,” “treatment,” or “treating” as used herein refers to therapeutic treatment, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. The terms “treat,” “treated,” “treatment,” or “treating” as well as words stemming therefrom, as used herein, do not necessarily imply 100% or complete treatment. Rather, there are varying degrees of treatment of which one of ordinary skill in the art recognizes as having a potential benefit or therapeutic effect. In this respect, the disclosed methods can provide any amount of any level of treatment of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

The term “biological sample,” as used herein, generally refers to a sample derived from or obtained from a subject, such as a mammal (e.g., a human). Biological samples are contemplated to include but are not limited to, hair, fingernails, skin, sweat, tears, ocular fluids, nasal swab or nasopharyngeal wash, sputum, throat swab, saliva, mucus, blood, serum, plasma, placental fluid, amniotic fluid, cord blood, emphatic fluids, cavity fluids, earwax, oil, glandular secretions, bile, lymph, pus, microbiota, meconium, breast milk, bone marrow, bone, CNS tissue, cerebrospinal fluid, adipose tissue, synovial fluid, stool, gastric fluid, urine, semen, vaginal secretions, stomach, small intestine, large intestine, rectum, pancreas, liver, kidney, bladder, lung, and other tissues and fluids derived from or obtained from a subject.

The term “tumor” or “tumor cells” as used herein, generally refers to cells that grow and divide more than they should or do not die when they should. In some cases, tumor cells are present in a solid mass, such as a solid tumor, or in some cases, tumor cells are found in a non-solid form, such as in blood cancers. Tumor or tumor cells also can include metastasis or metastasizing cells, where cancer cells break away from the original (primary) tumor and may form a new tumor in other organs or tissues of the body.

The term “ecDNA signature” as used herein, generally refers to one or more characteristics common to tumors or tumor cells that are ecDNA+. In some cases, the ecDNA signature is selected from the group consisting of a gene amplification; a p53 loss of function mutation; absence of microsatellite instability (MSI-H); a low level of PD-L1 expression; a low level of tumor inflammation signature (TIS); a low level of tumor mutational burden (TMB); an increased frequency of allele substitutions, insertions, or deletions (indels); and any combination thereof. In some cases, the ecDNA signature can include an increase in copy number (gene amplification) in conjunction with particular structural variations. In some cases, the ecDNA signature can include a focal amplification. In some cases, ecDNA signature includes a detection or identification of ecDNA using an imaging technology. In some cases, ecDNA signature does not include any imaging or direct detection of ecDNA.

### Compounds

Described herein are method of treating cancer in a subject in need thereof comprising administering to the subject a CHK1 inhibitor.

### Compound 1

In some embodiments, the CHK1 inhibitor is Compound 1, or a pharmaceutically acceptable salt thereof. Compound 1 is 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

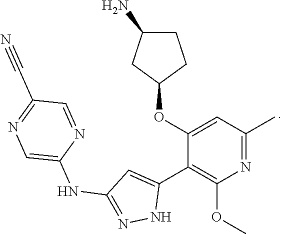 [SMILES: COc1nc(C)cc(O[C@@H]2CC[C@H](N)C2)c1-c1cc(Nc2cnc(C#N)cn2)n[nH]1]

In some embodiments, Compound 1 is a freebase. In some embodiments, Compound 1 is in the form of a salt.

### Crystalline Compound 1

Disclosed herein is a crystalline form of 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

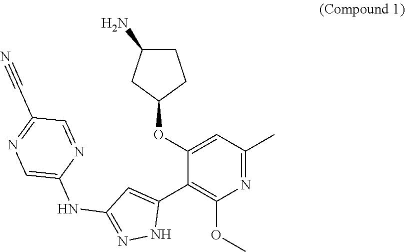 [SMILES: COc1nc(C)cc(O[C@@H]2CC[C@H](N)C2)c1-c1cc(Nc2cnc(C#N)cn2)n[nH]1]

or a pharmaceutically acceptable salt or solvate thereof.

Also disclosed herein is a crystalline form of freebase 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

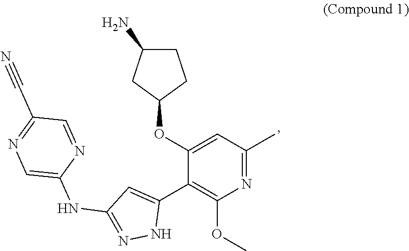 [SMILES: COc1nc(C)cc(O[C@@H]2CC[C@H](N)C2)c1-c1cc(Nc2cnc(C#N)cn2)n[nH]1]

or a pharmaceutically acceptable solvate thereof.

Also disclosed herein is a crystalline form of anhydrous freebase 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

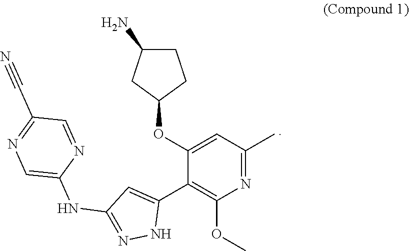 [SMILES: COc1nc(C)cc(O[C@@H]2CC[C@H](N)C2)c1-c1cc(Nc2cnc(C#N)cn2)n[nH]1]

Also disclosed herein is a crystalline form of the HCl salt of 5-((5-(4-(((1R,3S)-3-aminocyclopentyl)oxy)-2-methoxy-6-methylpyridin-3-yl)-1H-pyrazol-3-yl)amino)pyrazine-2-carbonitrile (Compound 1), or a pharmaceutically acceptable solvate thereof.

Also disclosed herein is a crystalline form of the HCl salt of 5-((5-(4-(((1R,3S)-3-aminocyclopentyl)oxy)-2-methoxy-6-methylpyridin-3-yl)-1H-pyrazol-3-yl)amino)pyrazine-2-carbonitrile (Compound 1).

Also disclosed herein is a crystalline form of the maleate salt of 5-((5-(4-(((1R,3S)-3-aminocyclopentyl)oxy)-2-methoxy-6-methylpyridin-3-yl)-1H-pyrazol-3-yl)amino)pyrazine-2-carbonitrile (Compound 1), or a pharmaceutically acceptable solvate thereof.

Also disclosed herein is a crystalline form of the maleate salt of 5-((5-(4-(((1R,3S)-3-aminocyclopentyl)oxy)-2-methoxy-6-methylpyridin-3-yl)-1H-pyrazol-3-yl)amino)pyrazine-2-carbonitrile (Compound 1).

Also disclosed herein is a crystalline form of the fumarate salt of 5-((5-(4-(((1R,3S)-3-aminocyclopentyl)oxy)-2-methoxy-6-methylpyridin-3-yl)-1H-pyrazol-3-yl)amino)pyrazine-2-carbonitrile (Compound 1), or a pharmaceutically acceptable solvate thereof.

Also disclosed herein is a crystalline form of the fumarate salt of 5-((5-(4-(((1R,3S)-3-aminocyclopentyl)oxy)-2-methoxy-6-methylpyridin-3-yl)-1H-pyrazol-3-yl)amino)pyrazine-2-carbonitrile (Compound 1).

Also disclosed herein is a crystalline form of the citrate salt of 5-((5-(4-(((1R,3S)-3-aminocyclopentyl)oxy)-2-methoxy-6-methylpyridin-3-yl)-1H-pyrazol-3-yl)amino)pyrazine-2-carbonitrile (Compound 1), or a pharmaceutically acceptable solvate thereof.

Also disclosed herein is a crystalline form of the citrate salt of 5-((5-(4-(((1R,3S)-3-aminocyclopentyl)oxy)-2-methoxy-6-methylpyridin-3-yl)-1H-pyrazol-3-yl)amino)pyrazine-2-carbonitrile (Compound 1).

Also disclosed herein is a crystalline form of the lactate salt of 5-((5-(4-(((1R,3S)-3-aminocyclopentyl)oxy)-2-methoxy-6-methylpyridin-3-yl)-1H-pyrazol-3-yl)amino)pyrazine-2-carbonitrile (Compound 1), or a pharmaceutically acceptable solvate thereof.

Also disclosed herein is a crystalline form of the lactate salt of 5-((5-(4-(((1R,3S)-3-aminocyclopentyl)oxy)-2-methoxy-6-methylpyridin-3-yl)-1H-pyrazol-3-yl)amino)pyrazine-2-carbonitrile (Compound 1).

Also disclosed herein is a crystalline form of the hippuric acid salt of 5-((5-(4-(((1R,3S)-3-aminocyclopentyl)oxy)-2-methoxy-6-methylpyridin-3-yl)-1H-pyrazol-3-yl)amino)pyrazine-2-carbonitrile (Compound 1), or a pharmaceutically acceptable solvate thereof.

Also disclosed herein is a crystalline form of the hippuric acid salt of 5-((5-(4-(((1R,3S)-3-aminocyclopentyl)oxy)-2-methoxy-6-methylpyridin-3-yl)-1H-pyrazol-3-yl)amino)pyrazine-2-carbonitrile (Compound 1).

Also disclosed herein is a crystalline form of the sulfate salt of 5-((5-(4-(((1R,3S)-3-aminocyclopentyl)oxy)-2-methoxy-6-methylpyridin-3-yl)-1H-pyrazol-3-yl)amino)pyrazine-2-carbonitrile (Compound 1), or a pharmaceutically acceptable solvate thereof.

Also disclosed herein is a crystalline form of the sulfate salt of 5-((5-(4-(((1R,3S)-3-aminocyclopentyl)oxy)-2-methoxy-6-methylpyridin-3-yl)-1H-pyrazol-3-yl)amino)pyrazine-2-carbonitrile (Compound 1).

### Freebase Crystalline Compound 1 Form FB-1

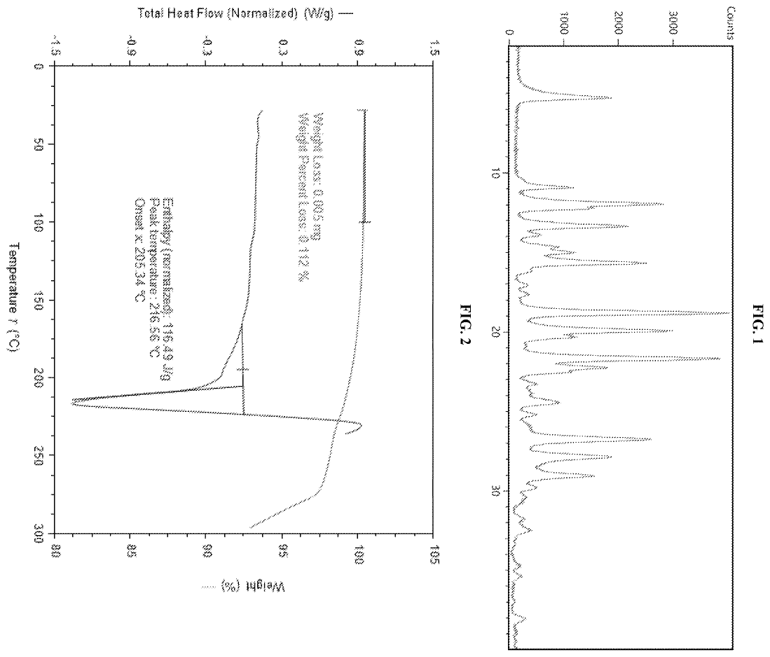

In some embodiments, the crystalline Compound 1 is freebase Form FB-1 characterized as having at least one of the following properties:
(a) an X-ray powder diffraction (XRPD) pattern substantially the same as shown in FIG. 1; (b) an X-ray powder diffraction (XRPD) pattern with characteristic peak at 11.96±0.1° 2θ, 18.81±0.1° 2θ, 19.91±0.1° 2θ, 21.67±0.1° 2θ, and 26.75±0.1° 2θ;
(c) an X-ray powder diffraction (XRPD) pattern with characteristic peak at 13.87±0.1° 2θ and 17.06±0.1° 2θ;
(d) a Differential Scanning Calorimetry (DSC) thermogram with an endotherm having a peak temperature at about 216.5° C. (onset);
(e) a Thermogravimetric Thermal Analysis (TGA) thermogram substantially the same as shown in FIG. 2;
(f) a Thermogravimetric Thermal Analysis (TGA) thermogram exhibiting a mass loss of about 0.10% from the onset of heating up to approximately 100.0° C.; or
(g) combinations thereof.

In some embodiments, the crystalline form has an X-ray powder diffraction (XRPD) pattern substantially the same as shown in FIG. 1.

In some embodiments, crystalline freebase Compound 1, Form FB-1 has an X-ray powder diffraction (XRPD) pattern with characteristic peaks found in Table 1.

In some embodiments, crystalline freebase Compound 1, Form FB-1 has an X-ray powder diffraction (XRPD) pattern with characteristic peak at 11.96±0.1° 2θ, 18.81±0.1° 2θ, 19.91±0.1° 2θ, 21.67±0.1° 2θ, and 26.75±0.1° 2θ.

In some embodiments, crystalline freebase Compound 1, Form FB-1 has an X-ray powder diffraction (XRPD) pattern with characteristic peak at 13.87±0.1° 2θ and 17.06±0.1° 2θ.

In some embodiments, crystalline freebase Compound 1, Form FB-1 has an X-ray powder diffraction (XRPD) pattern with a characteristic peak at 13.87±0.1° 2θ.

In some embodiments, crystalline freebase Compound 1, Form FB-1 has an X-ray powder diffraction (XRPD) pattern with characteristic peak at 17.06±0.1° 2θ.

In some embodiments, the X-ray powder diffraction (XRPD) pattern further comprises a peak at 5.27±0.1° 2θ.

In some embodiments, the X-ray powder diffraction (XRPD) pattern further comprises a peak at 13.34±0.1° 2θ.

In some embodiments, the X-ray powder diffraction (XRPD) pattern further comprises a peak at 15.66±0.1° 2θ.

In some embodiments, the X-ray powder diffraction (XRPD) pattern further comprises a peak at 22.24±0.1° 2θ.

In some embodiments, the X-ray powder diffraction (XRPD) pattern further comprises a peak at 27.84±0.1° 2θ.

In some embodiments, crystalline freebase Compound 1, Form FB-1 has an X-ray powder diffraction (XRPD) pattern with a characteristic peak at 5.27±0.1° 2θ.

In some embodiments, crystalline freebase Compound 1, Form FB-1 has an X-ray powder diffraction (XRPD) pattern with a characteristic peak at 11.96±0.1° 2θ.

In some embodiments, crystalline freebase Compound 1, Form FB-1 has an X-ray powder diffraction (XRPD) pattern with a characteristic peak at 13.34±0.1° 2θ.

In some embodiments, crystalline freebase Compound 1, Form FB-1 has an X-ray powder diffraction (XRPD) pattern with a characteristic peak at 13.87±0.1° 2θ.

In some embodiments, crystalline freebase Compound 1, Form FB-1 has an X-ray powder diffraction (XRPD) pattern with a characteristic peak at 15.66±0.1° 2θ.

In some embodiments, crystalline freebase Compound 1, Form FB-1 has an X-ray powder diffraction (XRPD) pattern with a characteristic peak at 17.06±0.1° 2θ.

In some embodiments, crystalline freebase Compound 1, Form FB-1 has an X-ray powder diffraction (XRPD) pattern with a characteristic peak at 18.81±0.1° 2θ.

In some embodiments, crystalline freebase Compound 1, Form FB-1 has an X-ray powder diffraction (XRPD) pattern with a characteristic peak at 19.91±0.1° 2θ.

In some embodiments, crystalline freebase Compound 1, Form FB-1 has an X-ray powder diffraction (XRPD) pattern with a characteristic peak at 21.67±0.1° 2θ.

In some embodiments, crystalline freebase Compound 1, Form FB-1 has an X-ray powder diffraction (XRPD) pattern with a characteristic peak at 22.24±0.1° 2θ.

In some embodiments, crystalline freebase Compound 1, Form FB-1 has an X-ray powder diffraction (XRPD) pattern with a characteristic peak at 26.75±0.1° 2θ.

In some embodiments, crystalline freebase Compound 1, Form FB-1 has an X-ray powder diffraction (XRPD) pattern with a characteristic peak at 27.84±0.1° 2θ.

In some embodiments, crystalline freebase Compound 1, Form FB-1 has an X-ray powder diffraction (XRPD) pattern with characteristic peaks at 5.27±0.1° 2θ, 11.96±0.1° 2θ, 13.34±0.1° 2θ, 15.66±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

In some embodiments, crystalline freebase Compound 1, Form FB-1 has an X-ray powder diffraction (XRPD) pattern with at least two characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

In some embodiments, crystalline freebase Compound 1, Form FB-1 has an X-ray powder diffraction (XRPD) pattern with at least three characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

In some embodiments, crystalline freebase Compound 1, Form FB-1 has an X-ray powder diffraction (XRPD) pattern with at least four characteristic peaks selected from 5.27±0.1° 2θ, 11.96±0.1° 20, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

In some embodiments, crystalline freebase Compound 1, Form FB-1 has an X-ray powder diffraction (XRPD) pattern with at least five characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

In some embodiments, crystalline freebase Compound 1, Form FB-1 has an X-ray powder diffraction (XRPD) pattern with at least six characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

In some embodiments, crystalline freebase Compound 1, Form FB-1 has an X-ray powder diffraction (XRPD) pattern with at least seven characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

In some embodiments, crystalline freebase Compound 1, Form FB-1 has an X-ray powder diffraction (XRPD) pattern with at least eight characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

In some embodiments, crystalline freebase Compound 1, Form FB-1 has an X-ray powder diffraction (XRPD) pattern with at least nine characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

In some embodiments, crystalline freebase Compound 1, Form FB-1 has an X-ray powder diffraction (XRPD) pattern with at least ten characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

In some embodiments, crystalline freebase Compound 1, Form FB-1 has an X-ray powder diffraction (XRPD) pattern with at least 11 characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

In some embodiments, crystalline freebase Compound 1, Form FB-1 has a Differential Scanning Calorimetry (DSC) thermogram with an endotherm having a peak temperature at about 216.5° C. (onset).

In some embodiments, crystalline freebase Compound 1, Form FB-1 has a Thermogravimetric Thermal Analysis (TGA) thermogram substantially the same as shown in FIG. 2.

In some embodiments, crystalline freebase Compound 1, Form FB-1 has a Thermogravimetric Thermal Analysis (TGA) thermogram exhibiting a mass loss of about 0.10% from the onset of heating up to approximately 100.0° C.

In some embodiments, crystalline freebase Compound 1, Form FB-1 is physically and chemically stable.

In some embodiments, crystalline freebase Compound 1, Form FB-1 is chemically stable.

In some embodiments, crystalline freebase Compound 1, Form FB-1 is chemically and physically stable according to a four-week stability study in open and close dish at 25° C./60% RH.

In some embodiments, crystalline freebase Compound 1, Form FB-1 is chemically and physically stable according to a four-week stability study in open and close dish at 40° C./75% RH.

In some embodiments, crystalline freebase Compound 1, Form FB-1 is chemically and physically stable according to a four-week stability study in open and close dish at 60° C.

In some embodiments, crystalline freebase Compound 1, Form FB-1 is a highly crystalline.

In some embodiments, crystalline freebase Compound 1, Form FB-1 is high melting.

In some embodiments, crystalline freebase Compound 1, Form FB-1 is non-hygroscopic.

In some embodiments, crystalline freebase Compound 1, Form FB-1 is anhydrous.

TABLE 1
XRPD peaks for Compound 1, Form FB-1

| Pos. [°2θ] | Height [cts] |
|---|---|
| 5.2689 | 1721.23 |
| 10.9152 | 1031.53 |
| 11.9596 | 2676.08 |
| 12.2724 | 1304.46 |
| 13.3417 | 2032.81 |
| 13.8724 | 475.45 |
| 14.6530 | 790.23 |
| 14.9972 | 1055.56 |
| 15.6634 | 2375.42 |
| 16.2005 | 278.72 |
| 17.0631 | 248.19 |
| 17.6224 | 245.43 |
| 18.8104 | 3868.35 |
| 18.8875 | 2971.30 |
| 19.9107 | 2866.67 |
| 19.9804 | 2532.42 |
| 20.3148 | 1082.38 |
| 21.6700 | 3724.27 |
| 21.7540 | 3239.63 |
| 22.2460 | 1672.27 |
| 22.5535 | 956.87 |
| 23.2597 | 390.65 |
| 23.7411 | 344.08 |
| 24.4332 | 827.00 |
| 25.2058 | 410.71 |
| 25.9436 | 368.18 |
| 26.7522 | 2475.52 |
| 27.8439 | 1780.24 |
| 29.0461 | 1488.74 |
| 29.7896 | 434.34 |
| 30.3904 | 245.87 |
| 31.7607 | 214.59 |
| 32.4723 | 330.93 |
| 34.7211 | 131.57 |
| 35.3634 | 163.14 |
| 36.9462 | 41.66 |
| 38.0530 | 228.72 |
| 38.8825 | 50.48 |
| 39.4946 | 16.58 |
| 39.9154 | 15.77 |

### Freebase Crystalline Compound 1 Form FB-2

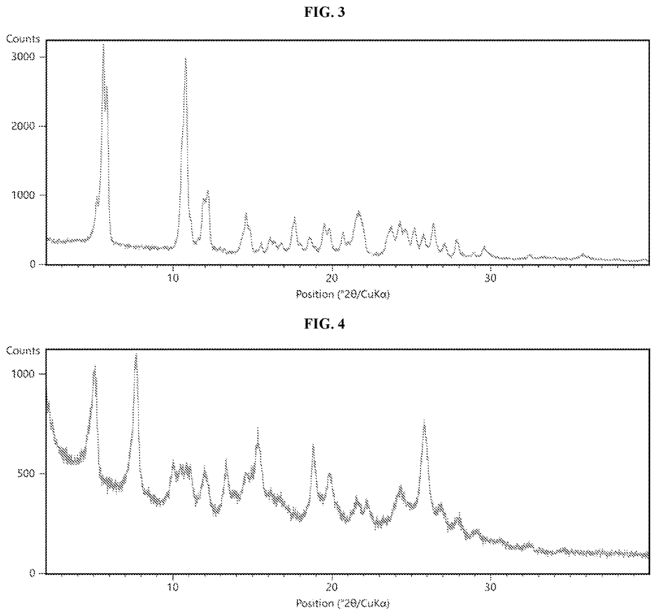

In some embodiments, the crystalline Compound 1 is freebase Form FB-2 characterized as having at least one of the following properties:
(a) an X-ray powder diffraction (XRPD) pattern substantially the same as shown in FIG. 3;
(b) an X-ray powder diffraction (XRPD) pattern with characteristic peaks found in Table 2; or
(c) combinations thereof.

In some embodiments, the crystalline form has an X-ray powder diffraction (XRPD) pattern substantially the same as shown in FIG. 3.

In some embodiments, crystalline freebase Compound 1, Form FB-2 has an X-ray powder diffraction (XRPD) pattern with characteristic peaks found in Table 2.

TABLE 2
XRPD peaks for Compound 1, Form FB-2

| Pos. [°2θ] | Height [cts] |
|---|---|
| 5.1826 | 675.90 |
| 5.5974 | 2942.36 |
| 5.8185 | 2266.51 |
| 10.5256 | 1532.83 |
| 10.7791 | 2830.43 |
| 11.9104 | 771.65 |
| 12.1745 | 872.39 |
| 14.5846 | 597.70 |
| 14.8452 | 319.30 |
| 15.5446 | 154.29 |
| 16.0902 | 224.18 |
| 16.3453 | 160.55 |
| 16.7944 | 144.43 |
| 17.6436 | 541.20 |
| 18.0390 | 113.78 |
| 18.5963 | 242.72 |
| 19.5111 | 449.17 |
| 19.8030 | 381.19 |
| 20.6793 | 338.64 |
| 21.6666 | 629.41 |
| 23.7183 | 421.38 |
| 24.2770 | 500.69 |
| 24.6158 | 402.52 |
| 25.1759 | 418.75 |
| 25.7456 | 328.47 |
| 26.3657 | 494.64 |
| 27.0642 | 182.36 |
| 27.8323 | 256.42 |
| 28.9163 | 76.84 |
| 29.5724 | 170.77 |
| 32.4407 | 61.34 |
| 33.5436 | 23.25 |
| 35.7841 | 65.09 |

### Freebase Crystalline Compound 1 Form FB-3

In some embodiments, the crystalline Compound 1 is freebase Form FB-3 characterized as having at least one of the following properties:
(a) an X-ray powder diffraction (XRPD) pattern substantially the same as shown in FIG. 4;
(b) an X-ray powder diffraction (XRPD) pattern with characteristic peaks found in Table 3; or
(c) combinations thereof.

In some embodiments, the crystalline form has an X-ray powder diffraction (XRPD) pattern substantially the same as shown in FIG. 4.

In some embodiments, crystalline freebase Compound 1, Form FB-3 has an X-ray powder diffraction (XRPD) pattern with characteristic peaks found in Table 3.

TABLE 3
XRPD peaks for Compound 1, Form FB-3

| Pos. [°2θ] | Height [cts] |
|---|---|
| 2.1645 | 513.43 |
| 5.0741 | 714.93 |
| 7.6606 | 768.53 |
| 10.0233 | 229.48 |
| 10.9317 | 200.07 |
| 12.0004 | 194.07 |
| 13.3506 | 229.16 |
| 14.5842 | 128.20 |
| 15.3364 | 325.93 |
| 18.8131 | 362.02 |
| 19.8363 | 235.05 |
| 21.5819 | 112.05 |
| 22.1752 | 109.12 |
| 24.3172 | 204.28 |
| 25.7942 | 548.56 |
| 26.8055 | 133.42 |
| 27.9234 | 87.12 |
| 29.0414 | 37.01 |
| 32.2051 | 13.47 |

Freebase Crystalline Compound 1 Form FB-4

In some embodiments, the crystalline Compound 1 is freebase Form FB-4 characterized as having at least one of the following properties:
(d) an X-ray powder diffraction (XRPD) pattern substantially the same as shown in FIG. 5;
(e) an X-ray powder diffraction (XRPD) pattern with characteristic peaks found in Table 4; or
(f) combinations thereof.

In some embodiments, the crystalline form has an X-ray powder diffraction (XRPD) pattern substantially the same as shown in FIG. 5.

In some embodiments, crystalline freebase Compound 1, Form FB-4 has an X-ray powder diffraction (XRPD) pattern with characteristic peaks found in Table 4.

TABLE 4
XRPD peaks for Compound 1, Form FB-4

| Pos. [°2θ] | Height [cts] |
|---|---|
| 5.5698 | 6558.39 |
| 10.4660 | 3824.20 |
| 10.7407 | 349.90 |
| 11.0873 | 373.12 |
| 11.7820 | 1001.58 |
| 12.7668 | 341.08 |
| 14.1434 | 211.45 |
| 14.4592 | 212.04 |
| 14.7497 | 563.13 |
| 15.2669 | 72.14 |
| 16.4416 | 221.01 |
| 16.7498 | 482.88 |
| 16.9783 | 600.44 |
| 18.0321 | 246.24 |
| 18.1262 | 505.73 |
| 18.9913 | 264.62 |
| 19.5605 | 368.79 |
| 19.7539 | 569.14 |
| 19.9335 | 377.90 |
| 20.4524 | 78.30 |
| 20.8689 | 285.87 |
| 20.9737 | 660.11 |
| 21.2354 | 636.26 |
| 21.5758 | 177.31 |
| 22.0157 | 217.26 |
| 22.3671 | 81.68 |
| 23.3936 | 862.28 |
| 23.4482 | 754.92 |
| 23.7136 | 197.39 |
| 24.0788 | 482.38 |
| 24.5242 | 522.32 |
| 25.0552 | 484.01 |
| 25.5906 | 352.23 |
| 26.2742 | 929.12 |
| 26.8415 | 111.57 |
| 27.0092 | 399.50 |
| 27.7784 | 411.58 |
| 28.5828 | 47.89 |
| 29.0766 | 60.31 |
| 29.4509 | 24.37 |
| 29.8135 | 30.93 |
| 30.9479 | 28.26 |
| 32.2923 | 62.13 |
| 32.9805 | 54.09 |
| 33.9390 | 24.81 |
| 36.7993 | 29.29 |
| 37.2360 | 32.54 |

### Freebase Crystalline Compound 1 Form FB-5

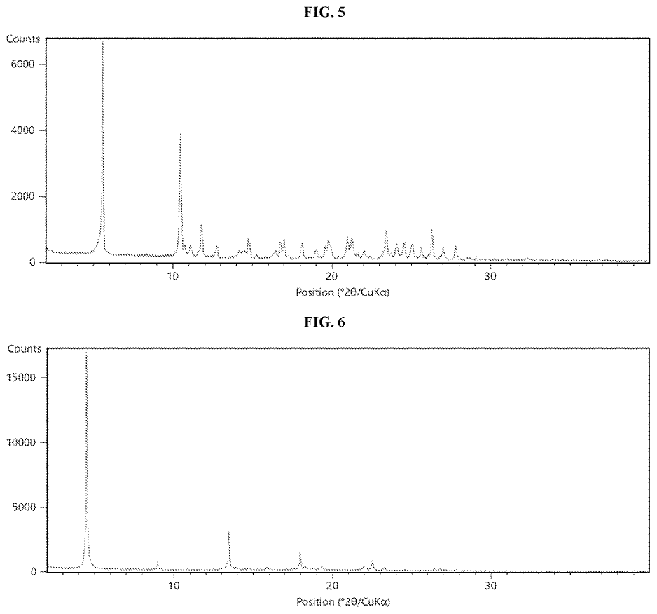

In some embodiments, the crystalline Compound 1 is freebase Form FB-5 characterized as having at least one of the following properties:
(g) an X-ray powder diffraction (XRPD) pattern substantially the same as shown in FIG. 6;
(h) an X-ray powder diffraction (XRPD) pattern with characteristic peaks found in Table 5; or
(i) combinations thereof.

In some embodiments, the crystalline form has an X-ray powder diffraction (XRPD) pattern substantially the same as shown in FIG. 6.

In some embodiments, crystalline freebase Compound 1, Form FB-5 has an X-ray powder diffraction (XRPD) pattern with characteristic peaks found in Table 5.

TABLE 5
XRPD peaks for Compound 1, Form FB-5

| Pos. [°2θ] | Height [cts] |
|---|---|
| 4.4816 | 17476.16 |
| 8.9601 | 611.40 |
| 10.8639 | 48.03 |
| 12.4728 | 61.76 |
| 12.8630 | 48.62 |
| 13.1357 | 193.14 |
| 13.4478 | 3006.94 |
| 14.6835 | 105.86 |
| 15.2813 | 89.21 |
| 15.8440 | 183.23 |
| 17.9559 | 1471.07 |
| 18.2569 | 311.92 |
| 18.7429 | 126.43 |
| 19.3413 | 299.53 |
| 21.9714 | 317.39 |
| 22.4908 | 766.33 |
| 23.2729 | 188.93 |
| 24.5310 | 62.76 |
| 26.4220 | 122.89 |
| 26.7392 | 141.31 |
| 27.0706 | 101.29 |
| 27.8403 | 145.62 |
| 29.6460 | 27.99 |
| 30.0811 | 44.63 |
| 31.0488 | 21.56 |
| 34.5055 | 19.53 |
| 38.9688 | 49.89 |

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising a compound described herein, or a pharmaceutically acceptable salt, solvate, tautomer, or stereoisomer thereof, and a pharmaceutically acceptable excipient.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as increased overall response rate, increased duration of response, more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal, epidural, or intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached.

### Methods

Disclosed herein is a method of treating cancer in a subject in need thereof comprising administering to the subject a pharmaceutical composition comprising Compound 1, or a pharmaceutically acceptable salt thereof. In some embodiments, the CHK1 inhibitor is Compound 1, or a pharmaceutically acceptable salt thereof.

In some embodiments of a method disclosed herein, the subject experiences a therapeutic response.

In some embodiments of a method disclosed herein, the therapeutic response comprises a reduction in the level of oncogene amplification in the tumor or tumor cells after treatment as compared to the level of oncogene amplification in the tumor or tumor cells prior to treatment.

In some embodiments of a method disclosed herein, the therapeutic response comprises reduction in of one or more of tumor growth, tumor size, number of tumor cells, or tumor metastasis as compared to prior to treatment.

In some embodiments of a method disclosed herein, the therapeutic response comprises a therapeutic benefit. In some embodiments of a method disclosed herein, the therapeutic benefit is stable disease (SD). In some embodiments of a method disclosed herein, the therapeutic benefit is partial response (PR). In some embodiments of a method disclosed herein, the therapeutic benefit is complete response (CR). In some embodiments of a method disclosed herein, a complete response is determined by RECISTv1.1 (or RANO for GBM). In some embodiments of a method disclosed herein, the therapeutic benefit is duration of response (DOR). In some embodiments of a method disclosed herein, the therapeutic benefit is progression-free survival (PFS). In some embodiments of a method disclosed herein, the therapeutic benefit is overall survival (OS).

In some embodiments of a method disclosed herein, the cancer comprises a solid tumor.

In some embodiments of a method disclosed herein, the cancer comprises a locally advanced or metastatic non-resectable solid tumor.

In some embodiments of a method disclosed herein, the cancer comprises a tumor or tumor cells harboring an oncogene amplification.

In some embodiments of a method disclosed herein, the oncogene amplification comprises an amplification of ABL, AKT1, AKT2, ALK, androgen receptor, BRAF, CCND1, CCND2, CCND3, CCNE1, CDK12, CDK4, CDK6, EGFR, ERBB2, EZH2, FGFR1, FGFR2, FGFR3, FLT3, IDH1/2, JAK2, JAK3, KIT, KRAS, MDM2, MDM4, MET, MYC, MYCL, MYCN, NRAS, PDGFRA, TERT, VEGFRA, or any combination thereof.

In some embodiments of a method disclosed herein, the oncogene amplification comprises an amplification of FGFR1, FGFR2, FGFR3 or a combination thereof. In some embodiments of a method disclosed herein, the oncogene amplification comprises an amplification of FGFR1, FGFR2, FGFR3 or a combination thereof and wherein the treatment further comprises administering an FGFR inhibitor.

In some embodiments of a method disclosed herein, the oncogene amplification comprises CDK4, CDK6, EGFR, FGFR1, FGFR2, or FGFR3.

In some embodiments of a method disclosed herein, the oncogene amplification comprises an amplification of EGFR. In some embodiments of a method disclosed herein, the oncogene amplification comprises an amplification of EGFR and wherein the treatment further comprises administering an EGFR inhibitor.

In some embodiments of a method disclosed herein, the oncogene amplification comprises an amplification of CDK4, CDK6, or a combination thereof. In some embodiments of a method disclosed herein, the oncogene amplification comprises an amplification of CDK4, CDK6, or a combination thereof and wherein the treatment further comprises administering a CDK4/6 inhibitor.

In some embodiments of a method disclosed herein, the oncogene amplification resides on ecDNA.

In some embodiments of a method disclosed herein, the oncogene amplification resides on one or more chromosomal loci.

In some embodiments of a method disclosed herein, the oncogene amplification is an ecDNA-derived amplification.

In some embodiments of a method disclosed herein, the oncogene amplification has a copy number of at least 6, at least 8, at least 10, at least 15, at least 20 or more than 20 copies of the oncogene or portion thereof.

In some embodiments of a method disclosed herein, the cancer includes malignant tumors whose size can be decreased, whose growth or spread can be slowed or halted, or whose symptom is in remission or alleviated, reduced, and/or completely cured by deleting or suppressing and/or inhibiting functions of CHK1. Malignant tumors of interest are, but not limited to, head and neck cancer, gastrointestinal cancer (esophageal cancer, gastric cancer, duodenal cancer, liver cancer, biliary tract cancer (gallbladder, bile duct cancer, etc.), pancreatic cancer, colorectal cancer (colon cancer, rectal cancer, etc.), etc.), lung cancer (non-small cell lung cancer, small cell lung cancer, squamous cell lung carcinoma, lung adenocarcinoma, mesothelioma, etc.), breast cancer, genital cancer (ovarian cancer, uterine cancer, cervical cancer, endometrial cancer, etc.), urinary cancer (kidney cancer, bladder cancer, prostate cancer, testicular tumor, etc.), hematopoietic tumors (leukemia, malignant lymphoma, multiple myeloma, etc.), bone and soft tissue tumors (e.g., soft tissue sarcomas, liposarcoma, and osteosarcomas), skin cancer, brain tumor (e.g., glioblastoma) and the like.

In some embodiments of a method disclosed herein, the term cancer is used in accordance with its plain ordinary meaning in light of the present disclosure and refers to all types of cancer, neoplasm or malignant tumors found in mammals, including leukemias, lymphomas, melanomas, neuroendocrine tumors, carcinomas, and sarcomas. Exemplary cancers that may be treated with a compound disclosed herein, or a pharmaceutically acceptable salt, solvate, tautomer, or stereoisomer thereof, pharmaceutical compositions include acute myeloid leukemia, adrenal cortical cancer, adrenal gland cancer, bladder cancer, bone cancer, brain cancer, breast cancer (e.g., ductal carcinoma, lobular carcinoma, primary, metastatic), breast cancer, cancer of the endocrine system, cancer of the hepatic stellate cells, cancer of the pancreatic stellate cells, cervical cancer, colon cancer, colorectal cancer, ductal carcinoma, endometrial cancer, esophageal cancer, gastric cancer, genitourinary tract cancer, glioblastoma, glioma, head and neck cancer, hepatocellular carcinoma, Hodgkin's Disease, kidney cancer, leukemia (e.g., lymphoblastic leukemia, chronic lymphocytic leukemia, hairy cell leukemia), liver cancer (e.g., hepatocellular carcinoma), lobular carcinoma, lung cancer (e.g., non-small cell lung carcinoma, squamous cell lung carcinoma, adenocarcinoma, large cell lung carcinoma, small cell lung carcinoma, carcinoid, sarcoma), liposarcoma, lymph node cancer, lymphoma (e.g., Mantel cell lymphoma, follicular lymphoma, diffuse large B-cell lymphoma, marginal zona lymphoma, Burkitt's lymphoma, Non-Hodgkin's Lymphoma), malignant carcinoid, malignant hypercalcemia, malignant pancreatic insulinoma, medullary thyroid cancer, medulloblastoma, melanoma, mesothelioma, multiple myeloma muscle cancer, neoplasms of the endocrine or exocrine pancreas, neuroblastoma, ovarian cancer, Paget's Disease of the Nipple, pancreatic cancer, papillary thyroid cancer, Phyllodes Tumors, premalignant skin lesions, primary thrombocytosis, prostate cancer (e.g. castration-resistant prostate cancer), renal carcinoma, rhabdomyosarcoma, salivary gland cancer, sarcoma, soft tissue sarcoma, squamous cell carcinoma (e.g., head, neck, or esophagus), stomach cancer, testicular cancer, thyroid cancer, urinary bladder cancer, or uterine cancer. In embodiments, the cancer is selected from bladder cancer, breast cancer, colon cancer, esophageal cancer, esophageal cancer, glioblastoma, liver cancer, lung cancer, ovarian cancer, pancreatic cancer, prostate cancer, salivary gland cancer, soft tissue sarcoma, squamous cell lung carcinoma, stomach cancer, and uterine cancer.

In some embodiments of a method disclosed herein, the cancer is an ovarian cancer. In some embodiments of a method disclosed herein, the ovarian cancer is a platinum resistant high-grade serous ovarian cancer, a primary peritoneal cancer, or a fallopian tube cancer.

In some embodiments of a method disclosed herein, the cancer is a uterine cancer. In some embodiments of a method disclosed herein, the uterine cancer is a high-grade endometrial carcinoma, a uterine serous carcinoma or a uterine carcinosarcoma.

In some embodiments of a method disclosed herein, the cancer is glioblastoma or a neuroblastoma.

In some embodiments of a method disclosed herein, the cancer is colorectal cancer, esophageal cancer, gastric cancer, gastroesophageal junction (GEJ) cancer, head and neck squamous cell carcinoma, liposarcoma, non-small cell lung cancer, or subtype squamous cell carcinoma.

In some embodiments of a method disclosed herein, the cancer is breast cancer or head, esophageal cancer, neck squamous cell carcinoma, non-small cell lung cancer, stomach cancer, or subtype squamous cell carcinoma.

In some embodiments of a method disclosed herein, the cancer is esophageal cancer, non-small cell lung cancer, a sarcoma, or stomach cancer. In some embodiments of a method disclosed herein, the cancer is esophageal cancer. In some embodiments of a method disclosed herein, the cancer is non-small cell lung cancer. In some embodiments of a method disclosed herein, the cancer is a sarcoma. In some embodiments of a method disclosed herein, the cancer is stomach cancer.

In some embodiments of a method disclosed herein, the subject has undergone one or more prior therapies.

In some embodiments of a method disclosed herein, the subject was non-responsive to the one or more prior therapies.

In some embodiments of a method disclosed herein, the subject developed resistance to the one or more prior therapies.

In some embodiments of a method disclosed herein, the one or more prior therapies is chemotherapies.

In some embodiments of a method disclosed herein, the one or more prior therapies is a PD1 antibody.

In some embodiments of a method disclosed herein, the one or more prior therapies is a PD-L1 antibody.

In some embodiments of a method disclosed herein, the one or more prior therapies is a CTLA4 checkpoint inhibitor.

In some embodiments of a method disclosed herein, the one or more prior therapies is VEGF targeting therapies (e.g., bevacizumab for ovarian cancer).

Disclosed herein is a method of treating cancer in a subject in need thereof comprising administering to the subject a pharmaceutical composition comprising Compound 1, or a pharmaceutically acceptable salt thereof, wherein the method further comprises obtaining a diagnostic indicator of oncogene amplification in a biological sample from the subject.

In some embodiments of a method disclosed herein, the diagnostic indicator is obtained prior to a first administration of Compound 1, or a pharmaceutically acceptable salt thereof.

In some embodiments of a method disclosed herein, the diagnostic indicator is obtained subsequent to a first administration of Compound 1, or a pharmaceutically acceptable salt thereof.

In some embodiments of a method disclosed herein, the diagnostic indicator is obtained subsequent to multiple administrations of Compound 1, or a pharmaceutically acceptable salt thereof.

In some embodiments of a method disclosed herein, the diagnostic indicator results from a next generation sequencing (NGS)-based assay.

In some embodiments of a method disclosed herein, the diagnostic indicator results from a fluorescence in situ hybridization (FISH) assay.

In some embodiments of a method disclosed herein, the diagnostic indicator comprises an indicator for ecDNA-derived oncogene amplification.

In some embodiments of a method disclosed herein, the diagnostic indicator is obtained from tumor or liquid biopsy.

In some embodiments of a method disclosed herein, the method further comprises assessing a sample from a subject for the presence or level of one or more of a gene amplification, a focal gene amplification, ecDNA, HSR, or an ecDNA signature.

In some embodiments of a method disclosed herein, the method further comprises obtaining information of the presence or level of one or more of a gene amplification, a focal gene amplification, ecDNA, HSR, or an ecDNA signature in the tumor or tumor cells from the subject prior to, during or subsequent to the administration of Compound 1, or a pharmaceutically acceptable salt thereof.

### Oncogene Amplification

Oncogene amplification-associated tumors are a segment of the cancer population with an extremely high unmet need. Patients whose cancers harbor high-copy oncogene amplification have significantly worse survival compared with the broader cancer population. Pan-cancer analysis of oncogene-amplified tumors, cross-referenced with the Surveillance, Epidemiology, and End Results program data, indicates that, in the US alone, this population represents more than 400,000 newly diagnosed cancer patients each year across multiple tumor types.

Despite the enormous health benefits and improvements in survival afforded by precision medicine and targeted therapies for cancer, these therapies have unfortunately proven largely non-efficacious in the oncogene amplification population. In addition, immune checkpoint inhibitors (e.g., pembrolizumab) might perform poorly in oncogene-amplified cancer populations and hyper-progression has been associated with oncogene-amplified tumor settings.

To date, HER2 inhibitors (e.g., trastuzumab) for HER2-overexpressing breast cancer, gastroesophageal junction cancer, and gastric cancer are the only targeted therapies approved in oncogene amplified (or overexpressed) cancer populations, with breast cancer being the only single agent approval. Targeted agents that have shown efficacy in patients whose cancers are driven by oncogene point mutations, gene fusions or skipping deletions have generally failed to demonstrate robust efficacy in patients whose tumors are driven by oncogene amplification. This lack of approved therapies is despite extensive clinical testing of targeted agents in oncogene-amplified cancer populations including EGFR inhibitors in EGFR-amplified glioblastoma multiforme, FGFR inhibitors in FGFR-amplified cancers, and CDK4/6 inhibitors in CDK4-amplified liposarcoma. These clinical data have resulted in the misconception that oncogene amplification may not be a relevant cancer driver. This erroneous conclusion is despite extensive data to the contrary. Recurrent focal copy number amplification and overexpression of established oncogene drivers (otherwise activated by mutation and/or gene fusion), as well as antitumor efficacy of targeted inhibition (genetic and pharmacologic) in short-term preclinical cancer models establishes that amplifications are drivers, even if targeted therapy treatment approaches are generally not translating to prolonged clinical benefit. However, the above disconnect suggests that cancers driven by oncogene amplifications are biologically different from other tumors and require a new therapeutic paradigm. Accordingly, improved understanding of oncogene amplification biology is necessary, with the objectives of advancing new therapeutic approaches, pharmaceutical targets, and new molecular entities for this high unmet need patient population.

### Role of Extrachromosomal DNA in Oncogene Amplification

Chromosomal instability and tumor heterogeneity have been suggested to account for many targeted therapy failures. Consistent with this hypothesis, oncogene amplification is a consequence of prior or ongoing chromosomal instability arising through either numerical and/or structural alterations in chromosomes and can give rise to ecDNA. It has long been recognized that oncogenes can be amplified not only on chromosomes but also on ecDNA, originally referred to as “double minutes.” However, the frequency, importance, and specific roles of ecDNA in cancer biology have not been well understood until recently.

Some of the most prevalent driver oncogenes are encoded on ecDNA and can confer a selective advantage to cancer cells. These oncogenes amplified on ecDNA have several features that distinguish them from chromosomally localized oncogene amplifications:
1. ecDNA lack centromeres, thus, in contrast to chromosomally localized amplification states, they segregate unequally into daughter cells during cell division. This property supports a non-Mendelian inheritance pattern, enabling extreme gene copy number changes in relatively few cell divisions and leads to extensive copy number heterogeneity driving adaptability and tumor evolution.
2. ecDNA are epigenetically dysregulated and contain accessible chromatin and hyper-transcribed gene regions that are often more actively expressed than chromosomally located genes.

These features distinguish ecDNA from other forms of oncogene amplification and facilitate a level of genomic plasticity and adaptability beyond chromosomal amplification that enable tumors to evade environmental insults, including targeted therapeutic pressure. New therapeutic approaches that interfere with ecDNA function are necessary to overcome ecDNA mediated adaptation in oncogene-amplified tumors.

ecDNA-enabled oncogene amplifications are a primary driver of oncogenesis, play a critical role in driving tumor heterogeneity, and enable cancer cells to rapidly become resistant to targeted oncogene therapies. ecDNA-enabled oncogene amplifications were observed in nearly half of all human cancer types but almost never found in normal cell. For example, ecDNA-enabled oncogene amplifications can be found in approximately 14% of primary cancer specimens and that more than half of all high-copy number oncogene amplifications (i.e., copy number value>8) reside on ecDNA. Further, many of the most aggressive tumor types contain the highest prevalence of ecDNA, including approximately 60% of glioblastoma multiforme and just under 50% of sarcomas.

Patients whose cancers harbor ecDNA experience significantly shorter survival than cancer patients whose tumors are driven by other molecular lesions, even when controlled for tumor type. These data strongly indicate that patients with ecDNA-enabled cancers require a new therapeutic paradigm to address this large unmet need.

### Role of Extrachromosomal DNA in Therapeutic Resistance

The unique features of ecDNA, coupled with the remarkable genome plasticity of ecDNA-enabled tumors, contribute to the tumor's aggressive nature and ability to evade therapeutic pressure via rapid genomic evolution. The first demonstration of therapeutic resistance driven by ecDNA was in a mouse cancer cell line whereby methotrexate treatment led to high amplification of dihydrofolate reductase (DHFR) on ecDNA, and which was lost upon removal of methotrexate. Similar instances of DHFR ecDNA amplification have been recapitulated in multiple human cancer cell lines. Furthermore, amplification of drug efflux pump genes on ecDNA, including the family of ABC transporters, has been observed to mediate resistance to various chemotherapies. An equivalent role for ecDNA in providing resistance to more current targeted therapies has also been well established. Evasion of therapeutic response to the EGFR inhibitor erlotinib is facilitated by rapid loss of the population of EGFRvIII amplifications on ecDNA in patient-derived glioblastoma multiforme cells, contemporaneous with occurrence of a new cell population containing MDM2 amplification on ecDNA; this observed effect is consistent with an equivalent lack of response to EGFR inhibitors in patients. Preclinical studies in a gastric cancer cell line containing FGFR2 amplified on ecDNA demonstrated that cellular resistance to the pan-FGFR inhibitor infigratinib could be driven by oncogene dependency switching from FGFR2 amplification on ecDNA to a new, rapid amplification of EGFR on ecDNA. Strikingly, this dependency was reversed back to FGFR2 amplification on ecDNA under EGFR inhibitory pressure via erlotinib. In each case, the initial cell population was sensitive to the respective targeted therapy, resulting in short lived anti-proliferative effects lasting several weeks. Resistance and regrowth to the targeted therapies occurred coincident with switching of the amplified oncogenes on ecDNA. The rapid rate of amplification change is unique to ecDNA and helps account for the intrinsic targeted therapy resistance of de novo oncogene-amplified cancers.

Similarly, mutant oncogenes (e.g., BRAFV600E and KRASG12C) can be amplified on ecDNA as a resistance mechanism to corresponding targeted therapies (e.g., BRAF/MEK or KRAS inhibitors). For example, a mutant BRAFV600E melanoma cell line developed ecDNA-enabled amplification of BRAFV600E after exposure to BRAF/MEK dual inhibition. This phenomenon has also been documented in clinical cases. Relatedly, numerous putative acquired resistance mechanisms to the KRASG12C inhibitor adagrasib have been reported and of these, a high-level focal amplification of KRASG12C on ecDNA, confirmed in vitro and in vivo, conferred resistance to both clinically validated KRASG12C inhibitors, adagrasib and sotorasib.

Collectively, these studies highlight the striking genomic plasticity and precipitous rise of ecDNA enabled oncogene amplification that enables cancer cells to adapt rapidly to therapeutic pressure. In conclusion, cancers driven by ecDNA-enabled oncogene amplification are biologically different from other oncogene activated tumors and require a new therapeutic paradigm.

### Dosing/Administration

Disclosed herein is a method of treating cancer in a subject in need thereof comprising administering to the subject a pharmaceutical composition comprising Compound 1, or a pharmaceutically acceptable salt thereof. In some embodiments, the pharmaceutical composition comprising Compound 1 comprises a crystalline form of Compound 1, a crystalline freebase form of Compound 1 or a crystalline anhydrous freebase form of Compound 1. In some cases, such form of Compound 1 has an X-ray powder diffraction (XRPD) pattern, a Differential Scanning Calorimetry (DSC) thermogram, a Thermogravimetric Thermal Analysis (TGA) thermogram or any combination thereof such as described herein.

### Rationale for Human Starting Dose of Compound 1

GLP-compliant repeat dose toxicology studies were conducted in rats and dogs following oral gavage Q2D with exposure duration of up to 29 days. The main effects observed in both species were related to bone marrow suppression/depletion and gastrointestinal toxicity, and were considered an on-target pharmacological effect of Compound 1 that is directly associated with CHK1 inhibition. Partial or complete reversibility of Compound 1-related major changes was demonstrated in all tissues, and there was no major unexpected toxicity identified.

In some embodiments of a method disclosed herein, Compound 1 is administered at a dose of about 10 mg to about 800 mg.

In some embodiments of a method disclosed herein, Compound 1 is administered at a dose of about 10 mg to about 400 mg.

In some embodiments of a method disclosed herein, Compound 1 is administered at a dose of about 10 mg to about 400 mg, whereby the subject experiences a therapeutic response.

In some embodiments of a method disclosed herein, the dose of compound 1 is between about 10 mg to about 20 mg, about 20 mg to about 40 mg, about 40 mg to about 80 mg, about 80 mg to about 120 mg, about 120 mg to about 160 mg, about 160 mg to about 200 mg, about 200 mg to about 400 mg.

In some embodiments of a method disclosed herein, the dose of compound 1 is between about 20 to about 40 mg, about 40 to about 80 mg, about 80 to about 120 mg, about 120 mg to about 160 mg, or about 160 mg to about 200 mg.

In some embodiments of a method disclosed herein, the dose of compound 1 is between about 10 mg to about 20 mg, about 20 mg to about 40 mg, about 40 to about 80 mg, or about 80 to about 120 mg.

In some embodiments of a method disclosed herein, the dose of compound 1 is between about 10 mg to about 20 mg. In some embodiments of a method disclosed herein, the dose of compound 1 is between about 20 mg to about 40 mg. In some embodiments of a method disclosed herein, the dose of compound 1 is between about 40 mg to about 80 mg. In some embodiments of a method disclosed herein, the dose of compound 1 is between about 80 mg to about 120 mg. In some embodiments of a method disclosed herein, the dose of compound 1 is between about 120 mg to about 160 mg. In some embodiments of a method disclosed herein, the dose of compound 1 is between about 160 mg to about 200 mg. In some embodiments of a method disclosed herein, the dose of compound 1 is between about 200 mg to about 400 mg.

In some embodiments of a method disclosed herein, the dose of compound 1 is about 10 mg, about 20 mg, about 40 mg, about 80 mg, about 120 mg, about 160 mg, or about 200 mg. In some embodiments of a method disclosed herein, the dose of compound 1 is about 10 mg. In some embodiments of a method disclosed herein, the dose of compound 1 is about 15 mg. In some embodiments of a method disclosed herein, the dose of compound 1 is about 20 mg. In some embodiments of a method disclosed herein, the dose of compound 1 is about 25 mg. In some embodiments of a method disclosed herein, the dose of compound 1 is about 30 mg. In some embodiments of a method disclosed herein, the dose of compound 1 is about 35 mg. In some embodiments of a method disclosed herein, the dose of compound 1 is about 40 mg. In some embodiments of a method disclosed herein, the dose of compound 1 is about 45 mg. In some embodiments of a method disclosed herein, the dose of compound 1 is about 50 mg. In some embodiments of a method disclosed herein, the dose of compound 1 is about 55 mg. In some embodiments of a method disclosed herein, the dose of compound 1 is about 60 mg. In some embodiments of a method disclosed herein, the dose of compound 1 is about 65 mg. In some embodiments of a method disclosed herein, the dose of compound 1 is about 70 mg. In some embodiments of a method disclosed herein, the dose of compound 1 is about 75 mg. In some embodiments of a method disclosed herein, the dose of compound 1 is about 80 mg. In some embodiments of a method disclosed herein, the dose of compound 1 is about 85 mg. In some embodiments of a method disclosed herein, the dose of compound 1 is about 90 mg. In some embodiments of a method disclosed herein, the dose of compound 1 is about 95 mg. In some embodiments of a method disclosed herein, the dose of compound 1 is about 100 mg. In some embodiments of a method disclosed herein, the dose of compound 1 is about 105 mg. In some embodiments of a method disclosed herein, the dose of compound 1 is about 110 mg. In some embodiments of a method disclosed herein, the dose of compound 1 is about 115 mg. In some embodiments of a method disclosed herein, the dose of compound 1 is about 120 mg. In some embodiments of a method disclosed herein, the dose of compound 1 is about 125 mg. In some embodiments of a method disclosed herein, the dose of compound 1 is about 130 mg. In some embodiments of a method disclosed herein, the dose of compound 1 is about 135 mg. In some embodiments of a method disclosed herein, the dose of compound 1 is about 140 mg. In some embodiments of a method disclosed herein, the dose of compound 1 is about 145 mg. In some embodiments of a method disclosed herein, the dose of compound 1 is about 150 mg. In some embodiments of a method disclosed herein, the dose of compound 1 is about 155 mg. In some embodiments of a method disclosed herein, the dose of compound 1 is about 160 mg. In some embodiments of a method disclosed herein, the dose of compound 1 is about 165 mg. In some embodiments of a method disclosed herein, the dose of compound 1 is about 170 mg. In some embodiments of a method disclosed herein, the dose of compound 1 is about 175 mg. In some embodiments of a method disclosed herein, the dose of compound 1 is about 180 mg. In some embodiments of a method disclosed herein, the dose of compound 1 is about 185 mg. In some embodiments of a method disclosed herein, the dose of compound 1 is about 190 mg. In some embodiments of a method disclosed herein, the dose of compound 1 is about 195 mg. In some embodiments of a method disclosed herein, the dose of compound 1 is about 200 mg.

In some embodiments of a method disclosed herein, the composition is administered orally.

In some embodiments of a method disclosed herein, the composition is administered parentally.

In some embodiments of a method disclosed herein, the composition is administered every day.

In some embodiments of a method disclosed herein, the composition is administered every other day.

In some embodiments of a method disclosed herein, the composition is administered on a cycle of day 1 and day 3 followed by a 4 day dosing holiday.

In some embodiments of a method disclosed herein, the composition is administered every 3 days or weekly.

In some embodiments of a method disclosed herein, the composition is administered every 3 days.

In some embodiments of a method disclosed herein, the composition is administered weekly.

In some embodiments of a method disclosed herein, the composition is administered with a dosing holiday of 4 days, 4-7 days, 7 days, or 14 days.

### Combination

Disclosed herein is a method of treating cancer in a subject in need thereof comprising administering to the subject a pharmaceutical composition comprising a Compound 1, or a pharmaceutically acceptable salt thereof and an additional therapeutic agent. In some embodiments, the CHK1 inhibitor is Compound 1, or a pharmaceutically acceptable salt thereof. In some embodiments, the pharmaceutical composition comprising Compound 1 comprises a crystalline form of Compound 1, a crystalline freebase form of Compound 1 or a crystalline anhydrous freebase form of Compound 1. In some cases, such form of Compound 1 has an X-ray powder diffraction (XRPD) pattern, a Differential Scanning Calorimetry (DSC) thermogram, a Thermogravimetric Thermal Analysis (TGA) thermogram or any combination thereof such as described herein.

In some embodiments of a method disclosed herein, the additional therapeutic agent is an EGFR inhibitor.

In some embodiments of a method disclosed herein, the EGFR inhibitor is selected from the group consisting of 602, 705, 707, abivertinib, ABX-900, afatinib, agerafenib (RXDX-105), alflutinib mesylate, amivantamab, APL-1898, ASK-120067, aumolertinib (almonertinib), BBT-176, BDTX-1535, BDTX-189, BEBT-109, befortinib mesylate, beitatini, BLU-701, BLU-945, BPI-361175, BPI-7711, BPI-D0316, C-005, CDP1, cetuximab, CH-7233163, CK-101, CMAB-017, dacomitinib, depatuxizumab, depatuxizumab mafodotin (ABT-414), DFP-17729, dositinib, DS-2087, DZD-9008, E01001, E-10C, epertinib, epitinib (HMPL-813), erlotinib, ES-072, FCN-411, FHND-9041, furmonertinib, FWD-1509, GB-263, GC-1118A, gefitinib, GMA-204, GR-1401, Hemay-022, HLX-07, HS-627, I-010, icotinib, imgatuzumab, IN-A008, JMT-101, JRF-103, JS-111, JS-113, JZB-28, KN-023, KN-026, KP-673, lapatinib, larotinib, lazertinib, LL-191, LYN 205, M1231, maihuatinib, marizomib, mobocertinib, MP-0274, MRG003, naputinib tosilate, nazartinib, necitumumab, neptinib, nimotuzumab, NRC-2694-A, NT-004, OBX1-012, olafertinib, olmutinib, ORIC-114, oritinib, osimertinib, panitumumab, pirotinib, poziotinib, PRB-001, pyrotinib, QL-1203, SCT-200, serclutamab, SHR-A1307, SIM-200, SPH-1188, SSGJ-612, SYN-004, TAD-011, tarloxotinib, TAS-6417, TGRX-360, theliatinib (HMPL-309), TPC-064, TQB-3804, TY-9591, WJ-13404, WSD-0922, XZP-5809, yinlitinib maleate, YK-029A, YZJ-0318, zorifertinib, and ZSP-0391.

In some embodiments of a method disclosed herein, the EGFR inhibitor is erlotinib. Erlotinib (TARCEVA6) is an oral small molecule inhibitor of the receptor tyrosine kinase EGFR. Early data showed anticancer activity in a several tumors including cancers of the lung and pancreas, and erlotinib was approved by the FDA in 2013 for EGFR mutant NSCLC with EGFR exon 19 deletions or exon 21 substitution mutations. Notably, erlotinib can inhibit wildtype EGFR and is not selective for mutant EGFR only.

The planned dosage of erlotinib is 150 mg orally once daily, given >1 hour before or 2 hours after food intake. This is the dose of erlotinib for treatment of NSCLC per the TARCEVA® United States Prescribing Information (USPI).

In some embodiments of a method disclosed herein, erlotinib is administered to the subject at a dose of 150 mg PO daily, 100 mg PO daily, or 50 mg PO daily.

In some embodiments of a method disclosed herein, the additional therapeutic agent is a FGFR inhibitor.

In some embodiments of a method disclosed herein, the FGFR inhibitor is selected from the group consisting of 3D-185, ABSK-011, ABSK-012, ABSK-061, ABSK-091, aldafermin, alofanib, AST-56100, AZD-4547, bemarituzumab, BFKB-8488A, BGS-2219, BIO-1262, BPI-17509, BPI-43487, CPL-304-110, derazantinib, E-7090, erdafitinib, EVER-4010001, EVT-601, FGF-401, fisogatinib, FPI-1966, futibatinib, gunagratinib, H3B-6527, HH-185, HMPL-453, HS-236, ICP-105, ICP-192, infigratinib, JAB-6000, KIN-3248, M-6123, MAX-40279, OM-RCA-001, pemigatinib, RLY-4008, rogaratinib, SAR-439115, SAR-442501, SC-0011, SY-4798, TT-00434, zoligratinib (FF-284), and WXSH-0011.

In some embodiments of a method disclosed herein, the FGFR inhibitor is pemigatinib. Pemigatinib (PEMAZYRE®) is an oral small molecule inhibitor of the receptor tyrosine kinase FGFR. Pemigatinib was first approved by the FDA in 2020 for previously treated, unresectable locally advanced or metastatic cholangiocarcinoma with a FGFR2 fusion or other rearrangement as detected by an FDA-approved test. Notably, pemigatinib can inhibit wildtype FGFR1, FGFR2, and FGFR3 receptors.

In some embodiments of a method disclosed herein, pemigatinib is administered to the subject at a dose of 13.5 mg PO daily, 9 PO mg, 4.5 mg PO daily and the dose is administered once daily for 14 days followed by 7 sequential days without administration of pemigatinib.

In some embodiments of a method disclosed herein, the FGFR inhibitor is futibatinib.

Futibatinib (LYTGOBIk) is indicated for the treatment of adults with previously treated, unresectable, locally advanced, or metastatic intrahepatic cholangiocarcinoma harboring fibroblast growth factor receptor 2 (FGFR2) gene fusions or other rearrangements. Futibatinib was approved for medical use in the United States in September 2022.

In some embodiments of a method disclosed herein, futibatinib is administered to the subject at a dose of 20 mg PO daily.

In some embodiments of a method disclosed herein, the additional therapeutic agent is a CDK4/6 inhibitor.

In some embodiments of a method disclosed herein, the CDK4/6 inhibitor is selected from the group consisting of abemaciclib, AG-122275, AM-5992, AT-7519, AU2-94, auceliciclib, BEBT-209, BPI-1178, BPI-16350, CS-3002, fascaplysin, FCN-437, FN-1501, GLR-2007, GW-491619, HEC-80797, HS-10342, IIIM-290, IIIM-985, lerociclib, milciclib maleate, MM-D37K, MS-140, NP-102, NUV-422, ON-123300, palbociclib, PF-06842874, PF-06873600, PF-07220060, QHRD-110, R-547, RGB-286199, RGT-419B, ribociclib, riviciclib, RO-0505124, SHR-6390, THR-53, THR-79, TQB-3303, TQB-3616, trilaciclib, TY-302, TY-302, voruciclib, VS2-370, WXWH-0240, XH-30002, and XZP-3287.

In some embodiments of a method disclosed herein, the CDK4/6 inhibitor is abemaciclib. Abemaciclib (VERZENIO6) is an oral small molecule inhibitor of CDK4/6. Abemaciclib was first approved by the FDA in 2017 for advanced or metastatic breast cancer, which is hormone receptor positive and HER-2 negative. Notably, abemaciclib can inhibit wildtype CDK4 and CDK6 receptors.

In some embodiments of a method disclosed herein, the abemaciclib is administered to the subject at a dose of about 50 mg twice daily, about 100 mg twice daily, or 150 mg twice daily.

In some embodiments of a method disclosed herein, the additional therapeutic agent is a BRAF inhibitor. In some embodiments of a method disclosed herein, the BRAF inhibitor is ABM-1310, agerafenib (RXDX-105), ARQ-736, ASN-003, AZ-304, AZ-628, BAL-3833, belvarafenib, BGB-3245, BI-882370, dabrafenib, DAY101, DP-2874, EBI-907, EBI-945, encorafenib, GDC-0879, lifirafenib, LUT-014, LYN 204, NMS-P285, NMS-P730, PF-04880594, PF-07284890, PLX-8394, RX-208, TL-241, UAI-201, UB-941, vemurafenib, VS-6766, or XL-281.

In some embodiments of a method disclosed herein, the additional therapeutic agent is a MDM2 or MDM4 inhibitor.

In some embodiments of a method disclosed herein, the MDM2 inhibitor is AD-021.32, ALRN-6924, APG-115, ASTX-295, ATSP-7041, BI-907828, CGM-097, CYC700, DS-5272, idasanutlin, KRT-232 (AMG-232), MD-224, MI-1061, MI-219, MI-43, MI-77301 (SAR405838, SAR299155), MK-8242, NU-8231, NVP-CGM097, OM-301, PXN-527, RAIN-32 (milademetan), RG7112 (R05045337), RG7388 (RG7775), Rigel-3, RO-2468, RO-5353, RO-5963, serdemetan (JNJ-26854165), SIL-43, siremadlin, or UBX-0101. In some embodiments of a method disclosed herein, the MDM4 inhibitor is17AAG, 489-PXN, ALRN-6924, APG-115, ATSP-7041, BI-907828, CTX1, FL-118, inulanolide A, K-178, or SAH-p53-8.

In some embodiments of a method disclosed herein, the additional therapeutic agent is a MET inhibitor.

In some embodiments of a method disclosed herein, the MET inhibitor is ABP-1130, BPI-1831, BPI-2021, BYON-3521, CG-203306, CX-1003, Debio-1144, EMD-94283, EMT-100, EMT-101, HE-003, LMV-12, LS-177, NX-125, OMO-2, PF-4254644, PRX-MET, PTX-2173, QBH-196, RP-1400, SAB-Y14, SAR-125844, SGX-126, SYD-3521, WXSH-0011, X-379, and XL-265, and anti-MET antibodies such as ABX-900, GB-263, FS-101, LY-3164530, LY-3343544, PMC-002, or SAIT-301. In some embodiments of a method disclosed herein, the MET inhibitor is ABN-401, ABT-700, AMG-208, AMG-337, ARGX-111, BAY-85-3474, BMS-817378, bozitinib, BPI-9016M, glumetinib, golvatinib tartrate, GST-HG161, HQP-8361, I-020, JNJ-38877605, kanitinib, merestinib, MK-2461, MK-8033, OMO-1, pamufetinib, S-49076, savolitinib, SPH-3348, tivantinib, SAR-125844, SCR-1515, and TPX-0022, or anti-MET antibodies such as APL-101, CKD-702, EMB-01, EMI-137, ficlatuzumab, HLX-55, HS-10241, MCLA-129, MT-8633, NOV-1105, RC-108, REGN-5093, SHR-A1403, Sym-015, or telisotuzumab vedotin. In some embodiments of a method disclosed herein, the MET inhibitor is amivantamab, capmatinib, crizotinib, or tepotinib.

In some embodiments of a method disclosed herein, the additional therapeutic agent is a KRAS inhibitor. In some embodiments of a method disclosed herein, the KRAS inhibitor is ABREVO1, ARS-1620, APG-1842, ATG-012, BBP-454, BEPT-607, BI-2852, BI-1823911, BPI-421286, BTX-2541, COTI-219, IMM-1811900, JAB-21000, JAB-22000, JAB-23000, JAB-BX300, JP-002, KR-12, LYN 202, MRTX-1133, RAS-F, RMC-6236, RMC-6291, SDGR 5, STX-301, and YL-15293, or anti-KRAS antibodies such as SBT-100, SBT-102, or SBT-300. In some embodiments of a method disclosed herein, the KRAS inhibitor is adagrasib, ARS-3248, D-1553, GDC-6036, JDQ-443, LY3537982, sotorasib (AMG 510), or BI 1701963.

### EXAMPLES

### Example 1: Solid State Characterization of Compound 1 Form Fb-1

Compound 1 Form FB-1 form crystalline XRPD pattern can be seen in FIG. 1. DSC analysis shows endotherm at 216.6° C. (Tₚₑₐₖ) with 0.11 wt % loss in TGA between RT to 100° C. (FIG. 2).

TABLE 6
Solid-state Characterization of Compound 1 Form FB-1

Material

| Properties | Lot#1 | Lot#2 |
|---|---|---|
| Appearance | Off-white powder | Off-white powder |
| DSC(Tpeak) | 216.6° C. | 216.5° C. |
| TGA (% wt loss) | 0.11 (RT-100° C.) | 0.08 (RT-100° C.) |
| % H₂O by oven KF | 0.2 | 0.46 |
| % IPA by GC | 1.185 | 1.626 |
| % THF by GC | 0.0 | 0.065 |
| % Cl by ion-chro | 0.06 | 0.10 |

### pKa Measurement

Material used in this study was FB-1. Dissociation constant of Compound 1 freebase was measured using potentiometric acid-base titration. Experiment procedure is described below. Measurement was done in triplicate suggesting pKa of 8.99±0.6 (SD). Experiment and predicted pKa values were similar.

### Procedure:

1) Weighed ~10 mg of starting material in 10 ml of MeOH
2) Added 25 mL of 0.5M NaCl
3) Adjusted pH value to around 3-4 with 0.1 M HCl
4) Titrated above solution with 0.5M NaOH solution from pH 3-12

TABLE 7
pKa of Compound 1 Form FB-1

| Compound 1 | MeOH | 0.5M NaCl | | |
|---|---|---|---|---|
| (mg) | (mL) | (mL) | Notes | pKa |
| 10.0 | 10 | 25 | Titrated with | 8.7 |
| 10.7 | 10 | 25 | 0.5M NaOH | 8.7 |
| 3.6 | 10 | 25 | | 9.7 |

### Water Activity Determination

Material used in this study was FB-1. Water activity of Compound 1 freebase was measured using Aqualab TDL-2. Experiment procedure is described below. Measurement was done in duplicate and material showed water activity of 0.47.

### Procedure:

1) Took approximately 3-5 mL of reference standard in sample dish
2) Set Aqualab TDL-2 temperature to 25° C.
3) Closed the sample chamber and start measurement
4) Confirmed reference solution values are within the standard limit
5) In new sample dish took approximately 50-100 mg of API and started the measurement

TABLE 8
Water activity of Compound 1 Form FB-1

| Material | Temp | Water activity | Mean | Notes |
|---|---|---|---|---|
| Std 0.15 | 25 | 0.157 | 0.156 | Instrument- |
| | | 0.155 | | Aqualab TDL 2 |
| Std 0.5 | | 0.510 | 0.507 | |
| | | 0.510 | | |
| Compound 1 | | 0.474 | 0.475 | |
| | | 0.475 | | |

### Partition Coefficient Determination by Shake Flask Method

The experimental Log P value was measured as Log P in DI water is 2.01±0.309 for 1 day (24 hours) with shake flask method. Due to the low solubility of Compound 1 product in DI water, slow stirring method was chosen for continuing the experiment, Log P in DI water is 1.64±0.259 for 5 day (120 hours). A general shake-flask Log P procedure and slow-stirring Log P procedure were used for the Log P determination. The average log P result is obtained from three repeat testing using HPLC quantitation.

### Procedure:

1) Partition coefficient was determined in 1-Octanol/aqueous phase conditions. (Aqueous phase was de-ionized water, and the ratio of 1-Octanol/aqueous phase was 50:50)
2) Before a partition coefficient is determined, the two solvents were mutually saturated at the temperature of the experiment. 500 mL of 1-Octanol and 500 mL of aqueous phase was transferred in a 1 L bottle and shake well for 24 hours, stand for at least 24 hours so that two phases can be separated.
3) Test condition—Approximately 10 mg of sample was dissolved in 10 mL of saturated 1-Octanol which was transferred 9 mL into a 20 mL glass sample vial containing 9 mL of aqueous phase. Triplicate for sample preparation.
4) The 20 mL glass sample vial was vigorously shaken for 24 hour using orbital shaker (BT Lab Systems, Model #BT909, MO, United States) at 200 rpm and then the flask was let stand for 2 hours so that system can reach the equilibrium. That was the 1-day (24 hours) sample. Continued shaken for 96 hours at 200 rpm and then the flask was let stand for 2 hours so that system can reach the equilibrium. That was the 5-days sample (120 hours). Solubility for Compound 1 in water is too low for using HPLC to define, so used 1-Octanol layer's result for Log P calculation.

TABLE 9
Compound 1 Log P 1-day (24 hours) result

| | | | Concentration | Concentration | | |
|---|---|---|---|---|---|---|
| | | | in | in 1- | | |
| | | | aqueous | Octanol | P = | |
| Exp | | Aqueous | layer | layer | O/W (n- | |
| ID | Notes | layer | (mg/ml)¹ | (mg/ml)² | octanol/water) | Log P |
| Time | Compound 1 T₀ | De-ionized | 0.0000 | 1.0020 | | |
| Zero | | water | | | | |
| 1 | Compound 1-1 | De-ionized | 0.0037 | 0.9983 | 269.8 | 2.43 |
| | 24 hours | water | | | | |
| 2 | Compound 1-2 | De-ionized | 0.0121 | 0.9899 | 81.8 | 1.91 |
| | 24 hours | water | | | | |
| 3 | Compound 1-3 | De-ionized | 0.0130 | 0.9890 | 76.1 | 1.88 |
| | 24 hours | water | | | | |

| | |
|---|---|
| Average ± SD | 2.01 ± |
| | 0.3 |

Note:
¹Compound 1 Aqueous layer concentration is defined by T₀ 1-Ocanol layer concentration - 1-Octanol layer samples 1-day (24 hours) concentration.
²Compound 1 in 1-Octanol layer concentration is defined by Compound 1 calibration curve.

TABLE 10
Compound 1 Log P 5-day (120 hours) result

| | | | Concentration | Concentration | | |
|---|---|---|---|---|---|---|
| | | | in aqueous | in 1-Octanol | P = | |
| Exp | | Aqueous | layer | layer | O/W (n- | Log |
| ID | Notes | layer | (mg/ml) | (mg/ml)¹ | octanol/water) | P |
| Time | Compound 1 T₀ | De-ionized | 0.0000 | 1.0019 | | |
| Zero | | water | | | | |
| 1 | Compound 1-1 | De-ionized | 0.0381 | 0.9638 | 25.3 | 1.40 |
| | 120 hours | water | | | | |
| 2 | Compound 1-2 | De-ionized | 0.0152 | 0.9867 | 64.9 | 1.81 |
| | 120 hours | water | | | | |
| 3 | Compound 1-3 | De-ionized | 0.0129 | 0.9890 | 76.7 | 1.88 |
| | 120 hours | water | | | | |

| | |
|---|---|
| Average ± SD | 1.64 ± |
| | 0.3 |

Note:
¹Compound 1 in 1-Octanol layer concentration is defined by Compound 1 calibration curve, and extra point 0.068 mg/mL added.

### REFERENCE

1. “Partition Coefficient (n-octanol/water): Shake Flask Method,” OECD Guideline 107, Jul. 27, 1995.
2. “Partition Coefficient (1-Octanol/Water): Slow-Stirring Method”, OECD Guideline 123, Mar. 23, 2006.

### Example 2: Polymorph Screening

### FB-1 Solubility Study

The approximate solubility of crystalline Compound 1 was estimated in 20 solvents and status/results are reported in Table 11.

### Procedure:

1) Weighed ~5 mg of starting material into a 4.0 mL vial. Add 25 μL of solvent at RT
2) Shook and stirred the solution. If no clear solution was obtained, added another 25 μL and repeated until 0.75 mL solvent added.
3) Afterwards, solvent was added in the increment of 0.25 mL up to 3.0 mL
4) Stirred the solution overnight

TABLE 11
Approximate solubility of Compound 1

| Exp | | Solvent | b.p | Solubility | Observa- |
|---|---|---|---|---|---|
| ID | Solvents | Class | (° C.) | (mg/mL) | tion |
| 1 | MeOH | 2 | 64.7 | S < 1.3 | Hazy |
| 2 | EtOH | 3 | 78.37 | S < 0.7 | Hazy |
| 3 | IPA | 3 | 82.5 | S < 1.1 | TSL |
| 4 | iPAc | 3 | 89 | S < 1.1 | TSL |
| 5 | EtOAc | 3 | 77.1 | S < 1.6 | TSL |
| 6 | nPA | 3 | 97 | S < 1.3 | TSL |
| 7 | ACN | 2 | 82 | S < 1.0 | TSL |
| 8 | MEK | 3 | 79.64 | 1.9 < S < 2.3 | CL |
| 9 | MIBK | 2 | 117-118 | S < 1.7 | TSL |
| 10 | MTBE | 3 | 55.2 | S < 1.2 | TSL |
| 11 | THF | 2 | 66 | 9.4 < S < 18.8 | CL |
| 12 | 1,4-Dioxane | 2 | 101 | 4.1 < S < 5.1 | CL |
| 13 | 2-MeTHF | 2 | 80.2 | 3.4 < S < 3.9 | CL |
| 14 | Heptane | 3 | 98.42 | S < 1.3 | TSL |
| 15 | Toluene | 2 | 110.6 | S < 1.9 | TSL |
| 16 | Water | — | 100 | S < 1.5 | TSL |
| 17 | Acetone | 3 | 56 | 5.5 < S < 7.3 | CL |
| 18 | DCM | 2 | 39.6 | 6.0 < S < 8.0 | CL |
| 19 | DMF | 2 | 153 | S > 118.0 | CL→SL* |
| 20 | DMSO | 3 | 189 | S > 100.0 | CL→SL* |

CL—clear;
TSL—Thin slurry;
*after overnight stirring

### Polymorph Screening of Compound 1 Form FB-1

Based on the approximate solubility of the starting material, a total of 80 polymorph screening experiments were completed using different screening techniques including anti-solvent addition, solid vapor diffusion, liquid vapor diffusion, slurry at RT, slurry at 50° C., slow evaporation, polymer induced crystallization, grinding, heat-cool-heat, and hydration experiments. Material used in this study was freebase Compound 1 Form FB-1.

### Procedure

1. Slurried at RT and 50° C. ~30 mg
   ~30 mg of API was slurried in 0.5-1.0 mL of different solvents in a 4.0 mL glass vial using magnetic stirrer at RT. The solids in slurry were characterized by XRPD after 5-7 days
2. Hydration at RT and 50° C.
   ~30 mg of API was slurried in 0.5-1.0 mL of different solvents in a 4.0 mL glass vial using magnetic stirrer at RT. The solids in slurry were characterized by XRPD after 5-7 days
3. Solid vapor diffusion
   ~30 mg of API was kept in a 4.0 mL glass vial; the vial was placed in 20 mL glass vial containing solvent. The solids were characterized by XRPD after 7-10 days
4. Anti-solvent addition
   ~30 mg of API was dissolved in solvent to create a saturated solution and anti-solvent was added up to 10 volume ratios, the obtained solids were characterized by XRPD
5. Liquid vapor diffusion
   ~30 mg of API was dissolved in solvent to create a saturated solution in a 4.0 mL glass vial, the vial was placed in 20 mL glass vial containing anti-solvent. The obtained solids were characterized by XRPD after 7-10 days
6. Polymer induced crystallization
   ~30 mg of API and 2.0 mg of listed polymer was added in solvent and kept stirring at RT. The solids in slurry or precipitated solids from solution were characterized by XRPD after 7-10 days
7. Grinding:
   Grind ~30 mg of API in mortar-pestle for few minutes. Afterwards analyze the grinded material by XPRD.
8. Compaction:
   ~100 mg of API was compressed at 20 kN using STYL'One Nano and B-type tooling. Afterwards tablet was crushed and powdered material was analyzed by XPRD.

TABLE 12
Results from polymorph screening by slurry conditioning at RT

| | Amt | Solvent | | |
|---|---|---|---|---|
| Solvent | (mg) | (mL) | Obs | XRPD |
| 1,4- | ~30 | 0.5 | SL | Essentially |
| dioxane | | | | matching w/FB-1 |
| 2-Butanol | | | SL | FB-1 |
| 2-MeTHF | | | SL | FB-1 |
| Acetic | | | SL→CL → LT → | CL |
| acid | | | CL →SE | |
| Anisole | | | SL | FB-1 |
| CHCl₃ | | | SL | FB-1 |
| CPME | | | SL | FB-1 |
| DCM | | | SL | FB-1 |
| Et₂O | | | SL | FB-1 |
| DMAc | | | CL → LT → CL →SE | FB-1 |
| DMSO | | | CL→Hazy→LT → TSL | FB-1 |
| Acetone | | | SL | FB-4 |
| PEG 200 | | | SL | Essentially |
| | | | | matching w/FB-1 |
| EtOH | | | SL | FB-1 |
| MIBK | | | SL | FB-1 |
| MTBE | | | SL | FB-1 |
| n-hexane | | | SL | FB-1 |
| Pyridine | | | SL | FB-1 |
| Toluene | | | SL | FB-1 |

CL—clear liquid;
SL—Slurry;
TSL—thin slurry;
LT—temp between 2-8° C.;
SE—solvent evaporation at RT

TABLE 13
Results from polymorph screening by slurry conditioning at 50° C.

| Solvent (v:v) | Amt (mg) | Solvent (mL) | Obs | XRPD |
|---|---|---|---|---|
| IPA | ~30 | 0.5 | SL | FB-1 |
| 2-Butanol | | | SL | FB-1 |
| MIBK | | | SL | FB-1 |
| IPAc | | | SL | FB-1 |
| CPME | | | SL | FB-1 |
| MeCN | | | SL | FB-1 |
| EtOH/n-Heptane (1:3) | | 0.4 | SL | FB-1 |
| 2-MeTHF/n-Heptane (1:5) | | 0.6 | SL | FB-1 |
| 1,4-Dioxane/Hexane (1:5) | | | SL | FB-1 |
| CHCl₃/n-Heptane (1:5) | | | SL | FB-1 |

SL—Slurry

TABLE 14
Results from polymorph screening by hydration at RT

| | Amt | Solvent | | |
|---|---|---|---|---|
| Solvent (1:1 v/v) | (mg) | (mL) | Obs | XRPD |
| Acetone/H₂O | ~30 | 0.5 | SL | FB-1 |
| Acetonitrile/H₂O | | | SL | FB-1 |
| DMF/H₂O | | | SL | Essentially matching |
| | | | | w/FB-1 |
| DMSO/H₂O | | | SL | Poor Xtal |
| Dioxane/H₂O | | | SL | FB-1 |
| EtOH/H₂O | | | SL | FB-1 |
| MeOH/IPA | | | SL | FB-1 |
| n-Propanol/H₂O | | | SL | FB-1 |
| THF/H₂O | | | SL | FB-1 |
| MeOH/H₂O | | | SL | FB-1 |

SL—Slurry

TABLE 15
Results from polymorph screening by hydration at 50° C.

| | Amt | Solvent | | |
|---|---|---|---|---|
| Solvent (1:1 v/v) | (mg) | (mL) | Obs | XRPD |
| Acetone/H₂O | ~30 | 0.5 | SL | FB-1 |
| Acetonitrile/H₂O | | | SL | FB-1 |
| DMF/H₂O | | | SL | Essentially matching |
| | | | | w/FB-1 |
| DMSO/H₂O | | | SL | Amorphous |
| Dioxane/H₂O | | | SL | FB-1 |
| EtOH/H₂O | | | SL | FB-1 |
| MeOH/IPA | | | SL | FB-1 |
| n-Propanol/H₂O | | | SL | FB-1 |
| THF/H₂O | | | SL | FB-1 |
| MeOH/H₂O | | | SL | FB-1 |

SL—Slurry

TABLE 16
Results from polymorph screening by anti-solvent addition at RT

| | Anti- | Anti-solvent | | |
|---|---|---|---|---|
| Solvent | solvent | (mL) | Observation | XRPD |
| THF | H₂O | 0.5 | Hazy | FB-1 |
| | MTBE | 0.75 | Hazy | Essentially |
| | | | | matching w/FB-1 |
| | n-Heptane | 0.25 | Hazy | FB-1 |
| NMP | IPAc | 3.0 | LT → CL →SE | FB-1 |
| | Anisole | 3.0 | LT → CL →SE | CL |
| | CPME | 3.0 | LT → CL →SE | CL |
| DMSO | H₂O | 0.075 | Hazy | Poorly crystalline |
| | Toluene | 1.0 | LT → CL →SE | FB-5 |
| | MTBE | 0.5 | Oil | FB-1 |

CL—clear liquid;
SL—Slurry;
LT—temp between 2-8° C.;
SE—solvent evaporation at RT

TABLE 17
Results from polymorph screening by solid vapor diffusion at RT

| Solvent | Observation | XRPD |
|---|---|---|
| 1-Butanol | Solids | FB-1 |
| Acetone | Solids | FB-4 |
| MEK | Solids | FB-1 |
| MTBE | Solids | FB-1 |
| 1,4-dioxane | Solids | FB-1 (extra peak at 4.5) |
| CHCl3 | Solids | FB-1 |

TABLE 18
Results from polymorph screening by liquid vapor diffusion at RT

| | | Anti- | | |
|---|---|---|---|---|
| | Anti- | solvent | | |
| Solvent | solvent | (mL) | Observation | XRPD |
| DMSO | EtOAc | 2.0 | CL→TSL | FB-1 |
| | MTBE | 2.0 | CL→TSL | FB-1 |
| | Toluene | 2.0 | CL→ LT → CL →SE | FB-5 |
| NMP | Hexane | 2.0 | CL→Not enough | Essentially |
| | | | solid to isolate → SE | matching w/FB-1 |
| | MTBE | 2.0 | CL→TSL | FB-1 |
| | IPAc | 2.0 | CL→TSL | Essentially |
| | | | | matching w/FB-1 |
| DMF | EtOAc | 2.0 | CL→TSL | FB-1 |
| | MTBE | 2.0 | CL→TSL | FB-1 |

CL—clear liquid;
TSL—thin slurry;
LT—temp between 2-8° C.;
SE—solvent evaporation at RT

TABLE 19
Results from polymorph screening by
polymer induced crystallization at RT

| Solvent | | Solvent | | |
|---|---|---|---|---|
| (90:10 v/v) | Polymer | (mL) | Observation | XRPD |
| MeOH/H₂O | PVP | 1.0 | SL | FB-1 |
| THF/H₂O | (2.0 mg) | 1.0 | CL→ LT → CL →SE | FB-1 |
| EtOH/H₂O | PVA | 1.0 | SL | FB-1 |
| THF/H₂O | (2.0 mg) | 1.0 | CL→ LT → CL →SE | FB-1 |
| Acetone/H₂O | PVC | 1.0 | CL→ LT → CL →SE | CL |
| | (2.0 mg) | | | |
| EtOH/H₂O | HPMC | 1.0 | SL | Essentially |
| | (2.0 mg) | | | matching |
| | | | | w/FB-1 |
| THF/H₂O | | 1.0 | CL→ LT → CL →SE | FB-1 |

CL—clear liquid;
SL—Slurry;
LT—temp between 2-8° C.;
SE—solvent evaporation at RT

Polymorph Form FB-4 was discovered from two test conditions i.e., slurry conditioning and solid vapor diffusion using acetone as a solvent system. DSC showed two endothermic events at 120.8 and 206.7° C. (Tpeak) with 4.4 wt % loss in TGA between RT-175° C. Thermal analysis data suggests Form FB-4 is likely hydrated form of the freebase.

Polymorph Form FB-5 was discovered from DMSO/toluene solvent system. Due to limited amount of material afforded from the test condition only DSC analysis was carried out which showed two endothermic events at 132.0 and 221.1° C. (Tpeak) suggesting most likely solvated or hydrated form of the freebase.

Form FB-1 was tested under trituration condition to understand potential of solid-to-solid phase transformation. However, XRPD data suggests no phase transition and material is stable under grinding condition. Form FB-1 was also tested for its potential to form hydrated form using aqueous-organic solvent system. End solids were analyzed by XRPD suggesting Form FB-1 is stable under these tested conditions.

Compound 1 hydrochloride salt break resulted in new polymorphic form referred to as Form FB-3. However, the end solids from multiple batches of salt break showed some variation in lower angles (20) in XRPD diffractograms. The solids were analyzed by DSC-TGA which suggests variation in XRPDs could be due to the different solvate content. Grinded and compacted material of Form FB-1 was analyzed by the XRPD, and data showed no solid-to-solid form change.

Eq. Solubility Study of Form FB-1

Experimental procedure and results are summarized in Table 20. Buffer media was prepared as per USP 35-NF 30 (Pg 5774) and biorelevant medias were prepared as per the instruction provided (biorelevant.com). ~40.0 mg of solids were added in 2.0 mL of media targeting to get slurry. System was set-up at 37° C. for ~18 h. Solution was tested for solubility by HPLC and pH.

Calibration curve was generated from 0.05 to 0.51 mg/mL concentration using Compound 1. Regression value was found to be >0.999. Calibration curve equation was further used for the solubility concentration determination.

TABLE 20
Eq. solubility data of Compound 1
Form FB-1 from biorelevant media

| | | | | Conc (mg/mL) | | |
|---|---|---|---|---|---|---|
| | | | | Rt = 6.9 | Rt = 7.5 | XRPD |
| | | pH | | min | min | of end |
| Media | Temp | Start | End | (API) | (Impurity) | solids |
| FaSSGF | 37 | 1.6 | 6.8 | 0.036 | ND | Mix of FB-1 |
| | | | | | | and HCl-2 |
| FaSSIF | | 6.4 | 8.2 | 0.000 | 0.014 | Mix of FB-1 |
| | | | | | | and HCl-2 |
| FeSSIF | | 4.9 | 6.4 | ND | 0.020 | Mix of FB-1 |
| | | | | | | and HCl-2 |
| Water | | 7.5 | 7.5 | 0.001 | ND | FB-1 |

ND—not detected

TABLE 21
Eq. solubility data of Compound 1 Form FB-1 from pH medias

| | | | | Conc | |
|---|---|---|---|---|---|
| | | pH | | (mg/mL) | |
| Media | Temp | Start | End | (API) | XRPD of end solids |
| 1.2 | 37 | 1.1 | 6.7 | 0.021 | HCl-2 |
| 3 | | 3.1 | 4.3 | 0.002 | Mix of FB-1 and HCl-2 |
| 4.5 | | 4.4 | 5.2 | 0.005 | Mix of FB-1 and HCl-2 |
| 6.8 | | 6.7 | 7.3 | 0.004 | Essentially matching |
| | | | | | w/#910-199-4 |

### Solid State Stability of Compound 1 Form Fm-1

Material used in this study was Compound 1 Form FB-1. Based on solid state and HPLC analysis Compound 1 Form FB-1 is stable for four weeks at 25° C./60% RH, 40° C./75% RH, and 60° C. Experiment results are summarized in Table 22.

TABLE 22
Compound 1 solid state stability study results/status

| Material and Equipment | | Note |
|---|---|---|
| API | FB-1 (Compound 1) | |
| Condition | 25° C./60% RH; 40° C./75% RH; 60° C. | |

| | |
|---|---|
| Equipment | Stability chamber |

Results/Status

| Exp ID (#1012-28) | Week | Vial Cap | Condition | XRPD | % A by HPLC |
|---|---|---|---|---|---|
| 1 | Week-1 | Open | 25° C./ | FB-1 | 97.55 |
| 2 | | Close | 60% RH | FB-1 | 97.63 |
| 3 | | Open | 40° C./ | FB-1 | 97.61 |
| 4 | | Close | 75% RH | FB-1 | 97.68 |
| 5 | | Open | 60° C. | FB-1 | 97.64 |
| 6 | | Close | | FB-1 | 97.58 |
| 7 | Week-2 | Open | 25° C./ | FB-1 | 97.50 |
| 8 | | Close | 60% RH | FB-1 | 97.43 |
| 9 | | Open | 40° C./ | FB-1 | 97.45 |
| 10 | | Close | 75% RH | FB-1 | 97.46 |
| 11 | | Open | 60° C. | FB-1 | 97.44 |
| 12 | | Close | | FB-1 | 97.35 |
| 13 | Week-4 | Open | 25° C./ | FB-1 | 97.50 |
| 14 | | Close | 60% RH | FB-1 | 97.43 |
| 15 | | Open | 40° C./ | FB-1 | 97.45 |
| 16 | | Close | 75% RH | FB-1 | 97.46 |
| 17 | | Open | 60° C. | FB-1 | 97.44 |
| 18 | | Close | | FB-1 | 97.35 |

### Example 2: Salt Screening

### Solubility Study

The approximate solubility of crystalline Compound 1 Form FB-1 was estimated in 20 solvents and status/results are reported in Table 23.

### Procedure:

1) Weigh ~5 mg of starting material into a 4.0 mL vial. Add 25 μL of solvent at RT
2) Shake and stir the solution. If no clear solution was obtained, add another 25 μL and repeat until 0.75 mL solvent added.
3) Afterwards, solvent was added in the increment of 0.25 mL up to 3.0 mL
4) Stir the solution overnight

TABLE 23
Approximate solubility of Compound 1 Form FB-1

| Exp | | Solvent | b.p | Solubility | |
|---|---|---|---|---|---|
| ID | Solvents | Class | (° C.) | (mg/mL) | Observation |
| 1 | MeOH | 2 | 64.7 | S < 1.3 | Hazy |
| 2 | EtOH | 3 | 78.37 | S < 0.7 | Hazy |
| 3 | IPA | 3 | 82.5 | S < 1.1 | TSL |
| 4 | iPAc | 3 | 89 | S < 1.1 | TSL |
| 5 | EtOAc | 3 | 77.1 | S < 1.6 | TSL |
| 6 | nPA | 3 | 97 | S < 1.3 | TSL |
| 7 | ACN | 2 | 82 | S < 1.0 | TSL |
| 8 | MEK | 3 | 79.64 | 1.9 < S < 2.3 | CL |
| 9 | MIBK | 2 | 117-118 | S < 1.7 | TSL |
| 10 | MTBE | 3 | 55.2 | S < 1.2 | TSL |
| 11 | THF | 2 | 66 | 9.4 < S < 18.8 | CL |
| 12 | 1,4-Dioxane | 2 | 101 | 4.1 < S < 5.1 | CL |
| 13 | 2-MeTHF | 2 | 80.2 | 3.4 < S < 3.9 | CL |
| 14 | Heptane | 3 | 98.42 | S < 1.3 | TSL |
| 15 | Toluene | 2 | 110.6 | S < 1.9 | TSL |
| 16 | Water | — | 100 | S < 1.5 | TSL |
| 17 | Acetone | 3 | 56 | 5.5 < S < 7.3 | CL |
| 18 | DCM | 2 | 39.6 | 6.0 < S < 8.0 | CL |
| 19 | DMF | 2 | 153 | S > 118.0 | CL→SL* |
| 20 | DMSO | 3 | 189 | S > 100.0 | CL→SL* |

CL—clear;
TSL—Thin slurry;
*after overnight stirring

### Salt Screening of Compound 1

Salt screening of crystalline Compound 1 was completed in 8 acids w/5 solvents at RT. Experimental procedure is described below. Experiment results are summarized in Table 24. Refer appendix II for detailed solid state characterization data.

### FB+Acid CI

Salt screening was set up with 8 counterions in 5 solvent systems. About 25 mg of the freebase was weighed to 2.0 mL vials. Counterions (CI) were added to the solids with 1:1 (APL:CI) molar ratio, and finally about 0.25 mL of solvents were added and sample vials were kept at RT with stirring on. Samples will be analyzed upon 3-5 days of stirring at RT for any solid-state changes by PXRD.

TABLE 24
Compound 1 salt screening results

| | IPA | MeCN | THF | EtOAc | Acetone |
|---|---|---|---|---|---|
| Acid | (A) | (B) | (C) | (D) | (E) |
| Blank | FB-1 | FB-1 | FB-1 | FB-1 | FB-2 |
| HCl | HCl-1 | HCl-2 | HCl-2 & | HCl-1 | HCl-2 |
| | | | FB-1 | | |
| H₂SO₄ | Poor crystalline, | SO₄-1 | Poor crystalline, essentially match SO4-1 | | |
| | essentially match | | | | |
| | SO₄-1 | | | | |
| MSA | MSA-1 | MSA-2 | MSA-1 | MSA-1 | MSA-1 |
| Maleic | Maleic-1 | Maleic-2 | Maleic-3 | Maleic- | Maleic-5 |
| acid | | | → Maleic- | 4→ | |
| | | | 6 | Maleic-7 | |
| Fumaric | Fumaric-1 | Fumaric-2 | Fumaric-3 | Fumaric- | Fumaric-1 |
| acid | | | → | 4 | |
| | | | Fumaric-5 | | |
| Citric acid | Amorphous | Citric-1 | Amorphous | FB-1 | Poor crystalline |
| L-Lactic acid | Lactic-1 | Lactic-1 | Lactic-2 | Lactic-1 | Lactic-3 |
| Hippuric acid | Hippuric-1 | Hippuric- | Hippuric-2 | Hippuric- | Poor crystalline |
| | | 1 | | 3 | |

TABLE 25
Compound 1 potential salt hits solid state characterization results

| | | | | | DVS |
|---|---|---|---|---|---|
| | | DSC ° C. | TGA % Wt | HNMR | (% wt H₂O gain |
| Acid | XRPD | (peak) | loss | (FB:acid) | between 0-90% RH) |
| NA | FB-1 | 219.0 | 0.0 (RT-150° | NA | NA |
| | | | C.) | | |
| HCl | HCl-1 | multiple | 3.8 (RT-100° | * | + |
| | | peaks | C.); | | |
| | | | 2.6 (100-225° | | |
| | | | C.) | | |
| | HCl-2 | 80.8, | 3.1 (RT-100° | * | + |
| | | 212.7 | C.) | | |
| | HCl-2 | 86.4, | 5.5 (RT-100° | * | + |
| | | 211.5, | C.) | | |
| | | 251.6 | | | |
| H₂SO₄ | SO₄-1 | 253.4 | 0 (RT-100° | * | + |
| | | | C.) | | |
| MSA | MSA-1 | 222.1 | 0 (RT-100° | * | + |
| | | | C.) | | |
| | MSA-1 | 205.9 | 0.2 (RT-100° | * | + |
| | | | C.) | | |
| Maleic | Maleic-1 | 216.0 | 0.94 (100-150° | 1:1 | 1.03 (No form |
| acid | | | C.) | | change) |
| | Maleic-2 | 75.0, | 1.5 (RT-100° | * | + |
| | | 187.4 | C.) | | |
| | Maleic-3 → | 60.5, | 1.1 (RT-100° | * | + |
| | Maleic-6 | 182.2 | C.) | | |
| Fumaric | Fumaric-3 → | 66.3, | 1.5 (RT-100° | * | + |
| acid | Fumaric-5 | 186.1 | C.) | | |
| | Fumaric-1 | 92.6, | 5.0 (RT-125° | * | + |
| | | 212.6 | C.) | | |
| L-lactic | Lactic-1 | 219.9 | 0.14 (RT-100° | 1:0.85 | + |
| acid | | | C.) | | |
| | Lactic-2 | 221.0 | 0.3 (RT-100° | 1:1 | + |
| | | | C.) | | |
| Hippuric | Hippuric-1 | 197.9 | 0 (RT-100° | 1:0.75 | + |
| acid | | | C.) | | |
| | Hippuric-2 | 197.5 | 0 (RT-100° | 1:0.9 | + |
| | | | C.) | | |

* Not tested;
+ Not in plan;
NA—Not applicable

### Salt Scale-Up and Characterization

### Hippuric-2

Hippurate salt scale up of crystalline Compound 1 was tested at sub-mg scale. Salt scale up experiment results are summarized in Table 26 and Table 27. Results indicate good reproducibility. Hippuric-2 was analyzed by DVS and data showed 0.740% wt H₂O gain between 0-80% RH, and 3.8% wt H₂O gain between 0-90% RH. However, post-DVS solids XRPD changed, referred to as mixture of hippuric-2 & hippuric-4.

TABLE 26
Salt scale up set-up

| | | | | | Start FB | Final FB | | |
|---|---|---|---|---|---|---|---|---|
| | FB | FB: | | Solvent | conc | conc | | |
| Acid | (mg) | acid | Solvent | (mL) | (mg/mL) | (mg/mL) | Temp | Observation |
| Hippuric | 400 | 1:1 | THF | 5.5 | 66.66 | 54.6 | RT | CL→SL |
| acid | | (Molar | | | | | | |
| | | eq.) | | | | | | |

SL—slurry;
CL—clear liquid;
RT—room temperature

TABLE 27
Salt scale up results

| | | | | HNMR | % A | |
|---|---|---|---|---|---|---|
| | | DSC ° C. | TGA % Wt | (FB: | (By | |
| Acid | XRPD | (peak) | loss | acid) | HPLC) | % Yield* |
| Hippuric | Hippuric-2 | 197.8 | 0.8 (RT- | 1:1 | 98.6 | 97.8 |
| | | | 100° C.) | | | |

*By solids recovery

### Maleic-1

Maleate salt scale up of crystalline Compound 1 was tested at sub-mg scale. Salt scale up results are summarized in Table 28 and Table 29. Results indicate good reproducibility in maleic-1 salt formation. Maleic-1 showed 1.03% wt H₂O gain between 0-90% RH, slightly hygroscopic. Post-DVS solids remained maleic-1.

TABLE 28
Salt scale up set-up

| | | | | | Start FB | Final FB | | |
|---|---|---|---|---|---|---|---|---|
| | FB | FB: | | Solvent | conc | conc | | |
| Acid | (mg) | acid | Solvent | (mL) | (mg/mL) | (mg/mL) | Temp | Observation |
| Maleic | 300 | 1:1 | IPA | 7.5 | 54.5 | 40.0 | RT | SL |
| acid | | (Molar | | | | | | |
| | | eq.) | | | | | | |

SL—slurry;
RT—room temperature

TABLE 29
Salt scale up results

| | | | | HNMR | % A | |
|---|---|---|---|---|---|---|
| | | DSC ° | TGA % Wt | (FB: | (By | |
| Acid | XRPD | C. (peak) | loss | acid) | HPLC) | % Yield* |
| Maleic | Maleic-1 | 214.9 | 0 (RT- | 1:1 | 98.83 | 96.4 |
| | | | 100° C.) | | | |

*By solids recovery;
^{†}assuming it is THF solvate

### Lactic-1 and Lactic-2

Test run at 25 mg of FB71-1 was performed to reproduce lactic-1. Results and status of salt scale up is summarized below. Test run was successful in reproducing lactic-1.

TABLE 30
Reproducibility check for lactic-1 from IPA

| | | | | | Start FB | Final FB | | |
|---|---|---|---|---|---|---|---|---|
| | FB | FB: | | Solvent | conc | conc | | |
| Acid | (mg) | acid | Solvent | (mL) | (mg/mL) | (mg/mL) | Temp | Observation |
| L-lactic | 25 | 1:1 | IPA | 0.7 | 50.0 | 35.7 | RT | SL→Cake→SL |
| acid | | (Molar | | | | | | |
| | | eq.) | | | | | | |

SL—slurry;
CL—clear liquid;
RT—room temperature

TABLE 31
Lactic-1 reproducibility study results

| | | DSC ° C. | TGA % Wt | HNMR |
|---|---|---|---|---|
| Acid | XRPD | (peak) | loss | (FB:acid) |
| L-lactic acid | Lactic-1 | 215.8 | 0.63 (RT-150° C.) | 1:0.86 |

Lactic salt scale up of crystalline Compound 1 is completed at 550 mg scale at RT. Results and status of salt scale up is summarized in Table 32 and Table 33. The reproducibility to crystallize lactic-1 was not good at 500 mg scale, and instead of lactic-1 form lactic-2 was afforded at the end of the study.

TABLE 32
Lactic salt scale up set-up

| | | | | | Start FB | Final FB | | |
|---|---|---|---|---|---|---|---|---|
| | FB | FB: | | Solvent | conc | conc | | |
| Acid | (mg) | acid | Solvent | (mL) | (mg/mL) | (mg/mL) | Temp | Observation |
| L-lactic | 550 | 1:1 | IPA | 15.4 | 50.0 | 35.7 | RT | SL |
| acid | | (Molar | | | | | | |
| | | eq.) | | | | | | |

SL—slurry;
CL—clear liquid;
RT—room temperature;
(IPA has 0.23 wt % of H₂O)

TABLE 33
Lactic salt scale up in IPA results

| | | | | HNMR | % A | |
|---|---|---|---|---|---|---|
| | | DSC ° C. | TGA % Wt | (FB: | (By | |
| Acid | XRPD | (peak) | loss | acid) | HPLC) | % Yield* |
| L-lactic | Lactic-6 | 221.2 | 0.7 (RT- | 1:0.95 | 98.2 | 92.4 |
| acid | (wet cake)→ | | 100° C.) | | | |
| | lactic-2 | | | | | |
| | (after | | | | | |
| | drying) | | | | | |

*By solids recovery

Lactic-2 salt was analyzed by DVS. Data showed 2.98% wt gain between 0-80% RH which is classified as moderately hygroscopic material. Between 0-90% RH material showed 3.66% wt gain, and post-DVS solids XRPD matched with lactic-2.

### Slurry Conditioning in H₂O

Freebase and selected 3-lead salts were slurried in water and kept for stirring at RT for 5-days. After 5-days of stirring end solids were analyzed by XRPD. In summary hippurtae, maleate, and lactate salt showed tendency to change to different solid form suggesting instability of these selected salt forms when slurry conditioned in water.

TABLE 34
XRPD results from slurry conditioning in H₂O

| Starting Form | End solid form after 5-day |
|---|---|
| FB-1 | FB-1 |
| Maleic-1 | Maleic-7 |
| Lactic-4 | Lactic-5 |
| Hippuric-2 | Hippuric-4 |

Eq. Solubility Study of FB-1, Hippuric-2, and Lactic-2

Experimental procedure and results are summarized in Table 35, Table 36, and Table 37. Buffer media was prepared as per USP 35-NF 30 (Pg 5774) and biorelevant medias were prepared as per the instruction provided (biorelevant.com). Approximate amount of solids were added in 0.5-1.0 mL of media targeting to get slurry. System was set-up at RT for 24 h. Afterwards, solids were filtered using centrifuge filter.

Solution was tested for solubility by HPLC and pH. The concentration of the solubility samples were too low, therefore the injection volume was adjusted from 0.3 μL to 3 μL. End solids were analyzed by XRPD.

TABLE 35
Freebase solubility test results

| FB-1 | | Media | Solubility | pH | | |
|---|---|---|---|---|---|---|
| (mg) | Media | (mL) | (μg/mL) | (±0.1)* | XRPD^{‡} | Notes |
| 37.1 | 1.2 | 1.0 | 8.5 | 2.52 | HCl-2 | SL |
| 32.5 | 4.5 | 1.0 | Not | 5.54 | Essentially | SL |
| | | | detected | | matching w/FB-2 | |
| 37.2 | 6.8 | 1.0 | 4.9 | 8.87 | New pattern | SL |
| 36.3 | FaSSGF | 1.0 | 2.9 | 7.65 | FB-1 + extra peaks | SL |
| | | | | | (2θ = 6.85, 12.48) | |
| 32.8 | FaSSIF | 1.0 | 12.5 | 8.01 | FB-1 + extra peak | SL |
| | | | | | (2θ = 6.76) | |
| 39.6 | FeSSIF | 1.0 | 1.2 | 8.10 | New pattern | SL |

*ML (equivalent to FB);
^{‡}End solids;
SL—slurry

TABLE 36
Hippuric-2 salt solubility test results

| Hippuric- | | Media | Solubility | pH | | |
|---|---|---|---|---|---|---|
| 2 (mg) | Media | (mL) | (μg/mL) | (±0.1)* | XRPD^{‡} | Notes |
| 41.3 | 1.2 | 1.0 | 37.7 | 7.86 | HCl-2 + extra peaks | SL |
| | | | | | (2θ = 13.0, | |
| | | | | | 16.7, 23.5) | |
| 46.0 | 4.5 | 1.0 | 26.8 | 4.95 | New pattern (matched | SL |
| | | | | | w/910-172-14) | |
| 41.4 | 6.8 | 1.0 | 22.1 | 7.25 | New pattern (matched | SL |
| | | | | | w/910-172-15) | |
| 40.7 | FaSSGF | 1.0 | 45.6 | 3.92 | HCl-2 + extra peaks | SL |
| | | | | | (2θ = 3.89, 15.37) | |
| 38.1 | FaSSIF | 1.0 | 2.1 | 6.60 | HCl-2 | SL |
| 38.0 | FeSSIF | 1.0 | 1.9 | 4.99 | HCl-2 + extra peaks | SL |
| | | | | | (2θ = 3.04, 9.43) | |

*ML (equivalent to FB);
^{‡}End solids;
SL—slurry

TABLE 37
Lactic-2 salt solubility test results

| Lactic- | | Media | Solubility | pH | | |
|---|---|---|---|---|---|---|
| 2 (mg) | Media | (mL) | (μg/mL) | (±0.1)* | XRPD^{‡} | Notes |
| 40.4 | 1.2 | 1.0 | 4.3 | 2.21 | Essentially | SL |
| | | | | | matching HCl-2 | |
| 41.1 | 4.5 | 1.0 | 7.7 | 5.26 | New pattern (matched | SL |
| | | | | | w/910-172-8) | |
| 45.2 | 6.8 | 1.0 | Not | 7.11 | New pattern (matched | SL |
| | | | detected | | w/910-172-9) | |
| 41.4 | FaSSGF | 1.0 | 25.7 | 3.94 | Essentially | SL |
| | | | | | matching HCl-2 | |
| 40.9 | FaSSIF | 1.0 | 10.0 | 6.48 | Essentially | SL |
| | | | | | matching HCl-2 | |
| 40.3 | FeSSIF | 1.0 | 4.1 | 4.96 | Essentially | SL |
| | | | | | matching HCl-2 | |

*ML (equivalent to FB);
^{‡}End solids;
SL—slurry

### Example 3: Salt Break

Salt break was conducted at 4.2 g scale. Experimental test results are summarized in Table 38.

TABLE 38
Salt break

| Material | | |
|---|---|---|
| Starting | | |
| material | Compound 1 HCl salt | Note |
| 1 | Add 4.2 g HCl salt to a 250 mL glass bottle | |
| 2 | Add 100 mL THF; place it on a magnetic stirrer | Slurry; THF- |
| | w/temp of 50° C. | 100 mL |
| 3 | Add sat. aq. Na₂CO₃ drop-wise to the solution | Slurry at start; |
| | and monitor the pH (10-11). Stop the reaction once | pH~10 |
| | pH is achieved. | |
| 4 | After 30 min; solids precipitation noticed | Expected to be NaCl |
| 5 | Separate THF layer, add extra THF to 250 mL glass | Total THF- |
| | bottle to rinse precipitated solids | 150 mL |
| 6 | To the filtered THF add anhydrous sodium sulfate | 1 h stirring |
| 7 | Filter the solution; and rinse the anhydrous | Total THF- |
| | sodium sulfate with extra THF. Strip down to | 200 mL |
| | solids using Rotavap | |
| 8 | Dry the isolated solids under vacuum at RT | |
| 9 | Analyze the dry solids with XRPD, NMR, HPLC, | Yellowish solids |
| | KF, DSC-TGA, GC | |

Results

| | |
|---|---|
| % A by HPLC | 97.33 |
| Crystal form by XRPD | Crystalline; New pattern matched with Form FB-3 |
| NMR | Matched with FB |
| Oven KF | 3.3% wt of H2O |
| GC | 0.98% of THF (9817 ppm) |
| % Yield | ~98 (By solids recovered; assuming anhydrous) |

### HPLC Method Development

During the course of salt screening, polymorph screening, and excipient compatibility studies multiple methods have been tested. For solubility study reported in this report following HPLC method was used to analyze the concentration of the Compound 1 (Table 39). HPLC method reported in Table 40 was used for the Log P and solid-state stability study.

TABLE 39
HPLC method information (used in eq. solubility study)
HPLC method details
Column: Supelco Assentis Express 90A C18,
150 × 2.1 mm, 2.7 μm, Cat#53825-U

| | |
|---|---|
| Mobile Phase A: 0.1% FA in water | Mobile Phase B: 0.1% FA in MeCN |
| Wavelength: 220 nm | Injection Volume: 3 μL |
| Diluent: DMSO/MeCN 20/80 | Nominal Conc.: 0.20 mg/mL |
| Column Temperature: 40° C. | Sample Temperature: ambient |

Gradient Elution Table

| Time (min) | A % | B % | Flow Rate (mL/min) |
|---|---|---|---|
| 0 | 95 | 5 | 0.3 |
| 4 | 75 | 25 | 0.3 |
| 8 | 25 | 75 | 0.3 |
| 12 | 5 | 95 | 0.3 |
| 12.1 | 95 | 5 | 0.3 |
| 18 | 95 | 5 | 0.3 |

TABLE 40
HPLC method information (used in Log P and solid-state stability study)
HPLC method details
Column: SKU: 186003021; XBridge BEH C18 Column,
130 Å, 3.5 μm, 2.1 mm × 50 mm

| Mobile Phase A: 0.05% TFA in water | Mobile Phase B: 0.05% TFA in MeCN |
|---|---|
| Wavelength: 254 nm +/− 4 nm | Injection Volume: 0.3 μL or 3.0 μL |
| Diluent: DMSO/MeCN 20/80 or | Nominal Conc.: 0.124 mg/mL |
| 100% DMSO (when hazy) | |
| Column Temperature: 45° C. | Sample Temperature: NA or 8° C. |
| | (only when stability needed) |

Gradient Elution Table

| Time (min) | A % | B % | Flow Rate (mL/min) |
|---|---|---|---|
| 0 | 100 | 0 | 0.8 |
| 7 | 40 | 60 | 0.8 |
| 8 | 0 | 100 | 0.8 |

### X-Ray Powder Diffraction (XRPD)

Instrument: Panalytical Empyrean
Parameters: X-Ray tube Cu (Kα radiation); Power: 45 kV×40 mA
Scanning range: 2 to 40 2θ (degree)
Step size: 0.01 degree
Scanning speed: 6.33 degree (2θ) per minute

### Thermogravimetric Analysis (TGA)

Instrument: TA Instruments Discovery TGA
Parameters: Ramp 10° C. per minute, 25 to 300° C., 50 mL/min N₂ sweep

### Differential Scanning Calorimetry (DSC)

Instrument: TA Instruments Discovery DSC
Parameters: Ramp 10° C. per minute, up to 300° C.

### Polarized Light Microscopy (PLM)

Instrument: Nikon Eclipse Ci POL
Camera: Nikon DS-Fi3
Software: Nikon NIS Elements

### ¹H NMR

Instrument: Bruker 400 MHz Ultrashield
Solvent: DMSO-d₆

### Water Activity Meter

Instrument: Aqualab TDL-2

### Titrator

Instrument: Mettler Toledo G20S Compact Potentiometric Titrator

### Dynamic Vapor Sorption (DVS)

Instrument: DVS Intrinsic, Surface Measurement Systems
Parameters: 25° C., 30-90-0-90-0% RH for 2 cycles

### High Performance Liquid Chromatography (HPLC)

| Parameter | Information |
|---|---|
| Column | XBridge BEH C18 Column, 130 A, 3.5 um, 2.1 mm*50 mm |
| Column Temp (° C.) | NA |
| Mobile Phase | H₂O (0.05% TFA)- MeCN (0.05% TFA) |
| | MeCN from 0 to 60% over 7 minutes |
| | 7-10 min, MeCN from 60% to 100% |
| Diluent | MeCN:DMSO (8:2) |
| Injection volume (μL) | 0.3 |
| Run time (min) | 10 |
| Standard conc (mg/mL) | 2.5 |
| Detection A (nm) | 254 |
| Flow rate (mL/min) | 0.8 |

The examples and embodiments described herein are for illustrative purposes only and in some embodiments, various modifications or changes are to be included within the purview of disclosure and scope of the appended claims.

What is claimed is:

1. A crystalline form of 5-((5-(4-(((1R,3S)-3-aminocyclopentyl)oxy)-2-methoxy-6-methylpyridin-3-yl)-1H-pyrazol-3-yl)amino)pyrazine-2-carbonitrile: 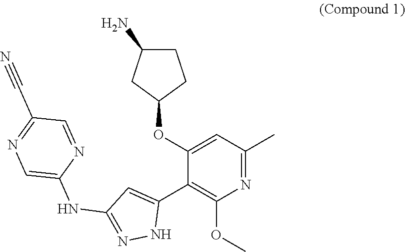 [SMILES: COc1nc(C)cc(O[C@@H]2CC[C@H](N)C2)c1-c1cc(Nc2cnc(C#N)cn2)n[nH]1]
or a pharmaceutically acceptable salt or solvate thereof.

2. A crystalline form of freebase 5-((5-(4-(((1R,3S)-3-aminocyclopentyl)oxy)-2-methoxy-6-methylpyridin-3-yl)-1H-pyrazol-3-yl)amino)pyrazine-2-carbonitrile: 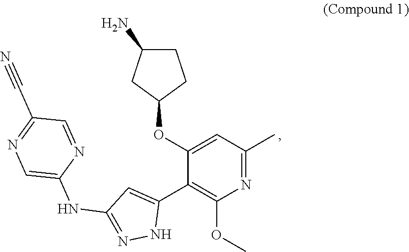 [SMILES: COc1nc(C)cc(O[C@@H]2CC[C@H](N)C2)c1-c1cc(Nc2cnc(C#N)cn2)n[nH]1]
or a pharmaceutically acceptable solvate thereof.

3. A crystalline form of anhydrous freebase 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

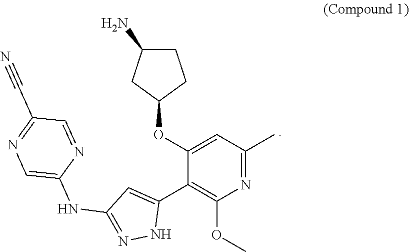 [SMILES: COc1nc(C)cc(O[C@@H]2CC[C@H](N)C2)c1-c1cc(Nc2cnc(C#N)cn2)n[nH]1]

4. The crystalline form of claim 1, wherein the crystalline Compound 1 is freebase Form FB-1 characterized as having at least one of the following properties:
(a) an X-ray powder diffraction (XRPD) pattern substantially the same as shown in FIG. 1;
(b) an X-ray powder diffraction (XRPD) pattern with characteristic peak at 11.96±0.1° 2θ, 18.81±0.1° 2θ, 19.91±0.1° 2θ, 21.67±0.1° 2θ, and 26.75±0.1° 2θ;
(c) an X-ray powder diffraction (XRPD) pattern with characteristic peak at 13.87±0.1° 2θ and 17.06±0.1° 2θ;
(d) a Differential Scanning Calorimetry (DSC) thermogram with an endotherm having a peak temperature at about 216.5° C. (onset);
(e) a Thermogravimetric Thermal Analysis (TGA) thermogram substantially the same as shown in FIG. 2;
(f) a Thermogravimetric Thermal Analysis (TGA) thermogram exhibiting a mass loss of about 0.10% from the onset of heating up to approximately 100.0° C.; or
(g) combinations thereof.

5. The crystalline form of any one of claims 1-4, wherein the crystalline form has an X-ray powder diffraction (XRPD) pattern substantially the same as shown in FIG. 1.

6. The crystalline form of any one of claims 1-5, wherein crystalline freebase Compound 1, Form FB-1 has an X-ray powder diffraction (XRPD) pattern with characteristic peaks found in Table 1.

7. The crystalline form of any one of claims 1-6, wherein crystalline freebase Compound 1, Form FB-1 has an X-ray powder diffraction (XRPD) pattern with characteristic peak at 11.96±0.1° 2θ, 18.81±0.1° 2θ, 19.91±0.1° 2θ, 21.67±0.1° 2θ, and 26.75 0.1° 2θ.

8. The crystalline form of any one of claims 1-6, wherein crystalline freebase Compound 1, Form FB-1 has an X-ray powder diffraction (XRPD) pattern with characteristic peak at 13.87±0.1° 2θ and 17.06±0.1° 2θ.

9. The crystalline form of any one of claims 1-6, wherein crystalline freebase Compound 1, Form FB-1 has an X-ray powder diffraction (XRPD) pattern with a characteristic peak at 13.87±0.1° 2θ.

10. The crystalline form of any one of claims 1-6, wherein crystalline freebase Compound 1, Form FB-1 has an X-ray powder diffraction (XRPD) pattern with characteristic peak at 17.06±0.1° 2θ.

11. The crystalline form of any one of claims 1-10, wherein the X-ray powder diffraction (XRPD) pattern further comprises a peak at 5.27±0.1° 2θ.

12. The crystalline form of any one of claims 1-11, wherein the X-ray powder diffraction (XRPD) pattern further comprises a peak at 13.34±0.1° 2θ.

13. The crystalline form of any one of claims 1-12, wherein the X-ray powder diffraction (XRPD) pattern further comprises a peak at 15.66±0.1° 2θ.

14. The crystalline form of any one of claims 1-13, wherein the X-ray powder diffraction (XRPD) pattern further comprises a peak at 22.24±0.1° 2θ.

15. The crystalline form of any one of claims 1-14, wherein the X-ray powder diffraction (XRPD) pattern further comprises a peak at 27.84±0.1° 2θ.

16. The crystalline form of any one of claims 1-5, wherein crystalline freebase Compound 1, Form FB-1 has an X-ray powder diffraction (XRPD) pattern with a characteristic peak at 5.27±0.1° 2θ.

17. The crystalline form of any one of claims 1-5, wherein crystalline freebase Compound 1, Form FB-1 has an X-ray powder diffraction (XRPD) pattern with a characteristic peak at 11.96±0.1° 2θ.

18. The crystalline form of any one of claims 1-5, wherein crystalline freebase Compound 1, Form FB-1 has an X-ray powder diffraction (XRPD) pattern with a characteristic peak at 13.34±0.1° 2θ.

19. The crystalline form of any one of claims 1-5, wherein crystalline freebase Compound 1, Form FB-1 has an X-ray powder diffraction (XRPD) pattern with a characteristic peak at 13.87±0.1° 2θ.

20. The crystalline form of any one of claims 1-5, wherein crystalline freebase Compound 1, Form FB-1 has an X-ray powder diffraction (XRPD) pattern with a characteristic peak at 15.66±0.1° 2θ.

21. The crystalline form of any one of claims 1-5, wherein crystalline freebase Compound 1, Form FB-1 has an X-ray powder diffraction (XRPD) pattern with a characteristic peak at 17.06±0.1° 2θ.

22. The crystalline form of any one of claims 1-5, wherein crystalline freebase Compound 1, Form FB-1 has an X-ray powder diffraction (XRPD) pattern with a characteristic peak at 18.81±0.1° 2θ.

23. The crystalline form of any one of claims 1-5, wherein crystalline freebase Compound 1, Form FB-1 has an X-ray powder diffraction (XRPD) pattern with a characteristic peak at 19.91±0.1° 2θ.

24. The crystalline form of any one of claims 1-5, wherein crystalline freebase Compound 1, Form FB-1 has an X-ray powder diffraction (XRPD) pattern with a characteristic peak at 21.67±0.1° 2θ.

25. The crystalline form of any one of claims 1-5, wherein crystalline freebase Compound 1, Form FB-1 has an X-ray powder diffraction (XRPD) pattern with a characteristic peak at 22.24±0.1° 2θ.

26. The crystalline form of any one of claims 1-5, wherein crystalline freebase Compound 1, Form FB-1 has an X-ray powder diffraction (XRPD) pattern with a characteristic peak at 26.75±0.1° 2θ.

27. The crystalline form of any one of claims 1-5, wherein crystalline freebase Compound 1, Form FB-1 has an X-ray powder diffraction (XRPD) pattern with a characteristic peak at 27.84±0.1° 2θ.

28. The crystalline form of any one of claims 1-5, wherein crystalline freebase Compound 1, Form FB-1 has an X-ray powder diffraction (XRPD) pattern with characteristic peaks at 5.27 0.1° 2θ, 11.96±0.1° 2θ, 13.34±0.1° 2θ, 15.66±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

29. The crystalline form of any one of claims 1-5, wherein crystalline freebase Compound 1, Form FB-1 has an X-ray powder diffraction (XRPD) pattern with at least two characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

30. The crystalline form of any one of claims 1-5, wherein crystalline freebase Compound 1, Form FB-1 has an X-ray powder diffraction (XRPD) pattern with at least three characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

31. The crystalline form of any one of claims 1-5, wherein crystalline freebase Compound 1, Form FB-1 has an X-ray powder diffraction (XRPD) pattern with at least four characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

32. The crystalline form of any one of claims 1-5, wherein crystalline freebase Compound 1, Form FB-1 has an X-ray powder diffraction (XRPD) pattern with at least five characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

33. The crystalline form of any one of claims 1-5, wherein crystalline freebase Compound 1, Form FB-1 has an X-ray powder diffraction (XRPD) pattern with at least six characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

34. The crystalline form of any one of claims 1-5, wherein crystalline freebase Compound 1, Form FB-1 has an X-ray powder diffraction (XRPD) pattern with at least seven characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

35. The crystalline form of any one of claims 1-5, wherein crystalline freebase Compound 1, Form FB-1 has an X-ray powder diffraction (XRPD) pattern with at least eight characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

36. The crystalline form of any one of claims 1-5, wherein crystalline freebase Compound 1, Form FB-1 has an X-ray powder diffraction (XRPD) pattern with at least nine characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

37. The crystalline form of any one of claims 1-5, wherein crystalline freebase Compound 1, Form FB-1 has an X-ray powder diffraction (XRPD) pattern with at least ten characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

38. The crystalline form of any one of claims 1-5, wherein crystalline freebase Compound 1, Form FB-1 has an X-ray powder diffraction (XRPD) pattern with at least 11 characteristic peaks selected from 5.27±0.1° 2θ, 11.96±0.1° 2θ, 13.34±0.1° 2θ, 13.87±0.1° 2θ, 15.66±0.1° 2θ, 17.06±0.1° 2θ, 18.81±0.1° 2θ, 19.91±0.1° 2θ, 21.67±0.1° 2θ, 22.24±0.1° 2θ, 26.75±0.1° 2θ, and 27.84±0.1° 2θ.

39. The crystalline form of any one of claims 1-38, wherein crystalline freebase Compound 1, Form FB-1 has a Differential Scanning Calorimetry (DSC) thermogram with an endotherm having a peak temperature at about 216.5° C. (onset).

40. The crystalline form of any one of claims 1-39, wherein crystalline freebase Compound 1, Form FB-1 has a Thermogravimetric Thermal Analysis (TGA) thermogram substantially the same as shown in FIG. 2.

41. The crystalline form of any one of claims 1-40, wherein crystalline freebase Compound 1, Form FB-1 has a Thermogravimetric Thermal Analysis (TGA) thermogram exhibiting a mass loss of about 0.10% from the onset of heating up to approximately 100.0° C.

42. The crystalline form of any one of claims 1-41, wherein crystalline freebase Compound 1, Form FB-1 is physically and chemically stable.

43. The crystalline form of any one of claims 1-42, wherein crystalline freebase Compound 1, Form FB-1 is chemically stable.

44. A pharmaceutical composition comprising the crystalline form of any one of claims 1-43 and a pharmaceutically acceptable excipient.

45. A method of treating cancer in a subject in need thereof comprising administering to the subject a crystalline form of any one of claims 1-43.

46. The method of claim 45, wherein the cancer comprises a solid tumor.

47. The method of claim 45, wherein the cancer comprises a locally advanced or metastatic non-resectable solid tumor.

48. The method of any one of claims 45-47, wherein the cancer comprises a tumor or tumor cells harboring an oncogene amplification.

49. The method of claim 48, wherein the oncogene amplification comprises an amplification of ABL, AKT1, AKT2, ALK, androgen receptor, BRAF, CCND1, CCND2, CCND3, CCNE1, CDK12, CDK4, CDK6, EGFR, ERBB2, EZH2, FGFR1, FGFR2, FGFR3, FLT3, IDH1/2, JAK2, JAK3, KIT, KRAS, MDM2, MDM4, MET, MYC, MYCL, MYCN, NRAS, PDGFRA, TERT, VEGFRA, or any combination thereof.

50. The method of claim 48 or claim 49, wherein the oncogene amplification resides on ecDNA.

51. The method of claim 48 or claim 49, wherein the oncogene amplification resides on one or more chromosomal loci.

52. The method of claim 48 or claim 49, wherein the oncogene amplification is an ecDNA-derived amplification.

53. The method of any one of claims 45-52, wherein the cancer is an ovarian cancer.

54. The method of claim 53, wherein the ovarian cancer is a platinum resistant high-grade serous ovarian cancer, a primary peritoneal cancer, or a fallopian tube cancer.

55. The method of any one of claims 45-52, wherein the cancer is a uterine cancer.

56. The method of claim 55, wherein the uterine cancer is a high-grade endometrial carcinoma, a uterine serous carcinoma or a uterine carcinosarcoma.

57. The method of any one of claims 45-52, wherein the cancer is colorectal cancer, esophageal cancer, gastric cancer, gastroesophageal junction (GEJ) cancer, head and neck squamous cell carcinoma, liposarcoma, non-small cell lung cancer, or subtype squamous cell carcinoma.

58. The method of any one of claims 45-52, wherein the cancer is a neuroblastoma.

59. The method of any one of claims 45-52, wherein the cancer is breast cancer, cholangiocarcinoma, esophageal cancer, neck squamous cell carcinoma, non-small cell lung cancer, stomach cancer, or subtype squamous cell carcinoma.

60. The method of any one of claims 45-52, wherein the cancer is esophageal cancer, non-small cell lung cancer, a sarcoma, or stomach cancer.

61. The method of any one of claim 45-60, wherein the treatment further comprises administering an additional therapeutic agent.

62. The method of claim 61, wherein the oncogene amplification comprises CDK4, CDK6, EGFR, FGFR1, FGFR2, or FGFR3.

63. The method of claim 62, wherein the treatment further comprises administering a CDK4/6 inhibitor, an EGFR inhibitor, or a FGFR inhibitor.

64. The method of claim 63, wherein the EGFR inhibitor is erlotinib.

65. The method of claim 63, wherein the FGFR inhibitor is pemigatinib.

66. The method of claim 63, wherein the FGFR inhibitor is futibatinib.

67. The method of claim 63, wherein the CDK4/6 inhibitor is abemaciclib.
